(19) United States
(12) Patent Application Publication
(10) Pub. No.: US 2009/0232733 A1
(43) Pub. Date: Sep. 17, 2009

(54) Diagnostic and Therapeutic Potential of Immune Globulin Intravenous (IGIV) Products

(76) Inventors: Brian O'Nuallain, Knoxville, TN (US); Rudi Hrncic, Orange Park, FL (US); Jonathan S. Wall, Knoxville, TN (US); Alan Solomon, Knoxville, TN (US)

Correspondence Address: MORGAN LEWIS & BOCKIUS LLP, 1111 PENNSYLVANIA AVENUE NW, WASHINGTON, DC, 20004, US

(21) Appl. No.: 11/911,506

(22) PCT Filed: Apr. 13, 2006
(86) PCT No.: PCT/US06/13858

§ 371 Date: Feb. 17, 2009

Related U.S. Application Data
(60) Provisional application No. 60/670,652, filed on Apr. 13, 2005.
(60) Provisional application No. 60/688,707, filed on Jun. 9, 2005.

(51) Int. Cl. A61K 51/00; A61K 49/00; A61B 5/055; A61B 8/00; A61K 39/395; C07K 1/16

(52) U.S. Cl. 424/1.49; 424/9.1; 424/9.34; 424/9.5; 424/130.1; 530/387.1

(57) ABSTRACT

The present invention provides IGIV and IGIV enriched for binding to amyloid fibrils and to partially denatured amyloidogenic precursor polypeptides. The present invention also provides methods for obtaining IGIV enriched for binding to amyloid fibrils and to partially denatured amyloidogenic precursor polypeptides. The IGIV recognizes amyloid fibrils and partially denatured amyloidogenic precursor polypeptides. They are useful for treating diseases and conditions associated with amyloid deposition. The IGIV of the present invention also are useful for diagnosing and detecting amyloid deposition.

### RELATED APPLICATIONS

This application claims the benefit of U.S. Provisional Application 60/670,652, filed Apr. 13, 2005, and U.S. Provisional Application 60/688,707, filed Jun. 9, 2005, which are herein incorporated by reference in their entirety.

### FIELD OF THE INVENTION

The present invention relates to the development of methods and tools effective for treating, preventing, and diagnosing amyloidosis. Specifically, the present invention is directed to methods of treating, preventing, and diagnosing amyloidosis comprising using antibodies.

### BACKGROUND OF THE INVENTION

Amyloidosis is a pathologic process in which normally soluble proteins of diverse chemical composition are deposited as fibrils in the brain, heart, liver, pancreas, kidneys, nerves, and other vital tissues, leading to organ failure and, eventually, death. This disorder represents an ever increasing, devastating medical and socioeconomic problem. Among the illnesses associated with amyloid are Alzheimer's disease, adult-onset (type 2) diabetes, certain forms of cancer (multiple myeloma and the related plasma cell disorder, primary [AL] amyloidosis) and inherited disorders (familial amyloidotic polyneuropathy, etc.), chronic inflammation (rheumatoid arthritis, tuberculosis, etc.), and the transmissible spongiform prion-associated encephalopathies. Additionally, amyloid deposition is an invariable consequence of aging (senile systemic amyloidosis, cataracts, etc.) (Benson et al., 2001; Ross et al., 2004; Enqvist et al., 2003; Meehan et al., 2004).

To date, 24 different amyloidogenic proteins have been identified (Table 1) (Westermark et al., 2002), but irrespective of their varied amino acid sequences, sources of origin, or biologic functions, all types of fibrils have virtually identical tinctorial and ultrastructural features, i.e., when stained by the diazobenzadine sulfonate dye Congo red and examined by polarizing microscopy, they exhibit a characteristic green birefringence (Westermark et al., 2002) and their interaction with thioflavin T (ThT) results in a 120 nm red shift in the excitation spectrum of this benzothiazole compound (LeVine et al., 1995).

TABLE 1
Amyloid Nomenclature: Amyloid fibril proteins and their precursors in humans*

| Amyloid | Protein | Syndrome or Involved Tissue |
|---|---|---|
| Protein | Precursor | (Systemic [S] or Localized [L] |
| AL | Immunoglobulin light chain | Primary (S,L), Myeloma-associated |
| AH | Immunoglobulin heavy chain | Primary (S,L), Myeloma-associated |
| ATTR | Transthyretin | Familial (S), Senile systemic, Tenosynovium |
| | | (L?) |
| Aβ₂M | β₂-microblobulin | Hemodialysis (S), Joints (L?) |
| AA | (Apo)serum AA | Secondary, reactive (S) |
| AapoAI | Apolipoprotein AI | Familial (S), Aortic (L) |
| AApo AII | Apolipoprotein AII | Familial (S) |
| Agel | Gelsolin | Familial (S) |
| Alys | Lyosozyme | Familial (S) |
| Afib | Fibrinogen α-chain | Familial (S) |
| Acys | Cystatin C | Familial (S) |
| Abri | ABriPP | Familial dementia, British (L, S?) |
| Adan | ADanPP | Familial dementia, Danish (L) |
| Aβ | Aβ protein precursor | Alzheimer's disease, aging (L) |
| AprP | Prion protein | Spongiform encephalopathies (L) |
| ACal | (Pro)calcitonin | C-cell thyroid tumors (L) |
| AIAPP | Islet amyloid polypeptide | Islets of Langerhans (L), Insulinomas |
| AANF | Atrial natriuetic factor | Cardiac atria (L) |
| APro | Prolactin | Aging pituitary (L), Prolactinomas |
| Alns | Insulin | latrogenic (L) |
| Amed | Lactadherin | Senile aortic, media (L) |
| AKer | Kerato-epithelin | Cornea; Familial (L) |
| A(Pin) | Unknown | Pindborg tumors (L) |
| ALac | Lactoferrin | Cornea; Familial (L) |

*Modified from Westermark et al., 2002

When negatively stained with uranyl acetate and viewed by electron microscopy, the fibrils are ˜10 nm in diameter, of indeterminate length, and consist of 2-5, often twisted, filaments arranged in parallel, with surface cross-banding patterns indicative of a helical structure (Goldsbury et al., 1997). Moreover, all types of amyloid fibrils have an x-ray fiber diffraction pattern that includes dominant structural repeat reflections at 4.7-4.8 Å on the meridian and spacings of 10 Å on the equator. These characteristics are consistent with a cross β-conformation and indicate that the amyloid polypeptide is organized, with respect to the fibril axis, as perpendicular β strands (Serpell et al., 2000). This cross-β pleated configuration (which has been confirmed by solid-state nuclear magnetic resonance [NMR] (Landsbury et al., 1995), Fourier transfer infrared [FTIR] spectroscopy (Seshadri et al., 1999), and x-ray crystallography (Makin et al., 2005) accounts for the typical birefringent and morphologic features of amyloid.

Polyclonal and monoclonal antibodies (mAb) have been generated that specifically recognize antigenic determinants expressed on amyloid fibrils or soluble oligomeric assembly intermediates, but not the native precursor proteins (Franklin et al., 1972; Linke et al., 1973; Gaskin et al., 1993; Gevorkian et al., 2004; Hrncic et al., 2000; O'Nuallain et al., 2002, 2004; Goldsteins et al., 1999; Kayed et al., 2003; Paramithiotis et al., 2003; Curin-Serbec et al., 2004; Dumoulin et al., 2004; Glabe et al, 2004). Additionally, IgG or IgM mAbs prepared against light chain (LC) or amyloid β peptide (Aβ) fibrils have been found to react with those formed from unrelated amyloidogenic precursors, including β₂-microglobulin (β₂M), serum amyloid A protein (SAA), islet amyloid polypeptide (IAPP), transthyretin (TTR), and polyglutamine (polyGln) (Hrncic et al., 2000; O'Nuallain et al., 2002). The demonstration that all fibrils, regardless of protein composition, share generic conformational epitopes has provided additional evidence for the presence of structural commonalities among these molecules.

In summary, amyloid is not a uniform deposit and may be composed of unrelated proteins. Various proteins have been identified as capable of forming amyloid in human diseases, for example, immunoglobulin light chains, serum amyloid A protein, β2-microglobulin, transthyretin, cystatin C variant, gelsolin, procalcitonin, PrP protein, amyloid β-protein, ApoA1, and lysozyme. Although these proteins are unrelated, the fibrils which they form have the following common biological properties: 1) they possess a β-pleated sheet secondary structure; 2) they are insoluble aggregates; 3) they exhibit green birefringence after Congo red staining; and 4) they possess a characteristic unbranching fibrillar structure when observed under an electron microscope.

Antibodies and IGIV

Antibodies are composed of heavy and light polypeptide chains which are joined by disulfide bridges. Antibodies are divided into different classes according to their heavy chain structure; antibodies belonging to the same class are referred to as isotypes of each other. In addition, antibodies of a given isotype can be divided into subtypes. Antigenic determinants on antibodies that differ among animals that have inherited different alleles are referred to as allotypes; antibodies that share an allotype are referred to as members of the same allotype. Another type of antigenic determinant present on antibody molecules are those found primarily in the hypervariable region of the antigen binding site of the antibody. These determinants are referred to as idiotypes; antibodies that share an idiotype are referred to as members of the same idiotype. Idiotypic determinants are controlled by both genetic and antigenic influences. Antibodies having common or shared idiotypes generally exhibit the same antigenic specificity. However, antibodies from genetically different individuals which share a common antigenic specificity may exhibit idiotypic heterogeneity but, in some instances, show a major cross-reactive antigenic determinant. Thus, antibodies which bind the same antigen may have distinct idiotypic determinants, but also may share cross-reacting properties.

Immune globulin intravenous (IGIV) products (also known as intravenous immunoglobulin (IVIG) or IVIg) are a highly purified IgG type preparation derived from the plasma of thousands of healthy blood donors. The spectrum of antibody specificity expressed is extremely large, and IGIV recognizes a large number of bacterial, viral and other infectious agents. In addition, these characteristics facilitated the use of IGIV for the combat of various infectious agents in immune deficient individuals. The anti-idiotypic activity of IGIV is utilized for the treatment of various autoimmune diseases, typically as an agent in diseases manifested by pathogenic idiotypic autoimmunity.

IGIV has been FDA approved to treat various diseases but not Alzheimer's or amyloidosis. IGIV has been used as an immunomodulatory agent in autoimmune diseases in aliogeneic bone marrow transplantation. IGIV has a well established effect in idiopathic thrombocytopenic purpura (ITP), Kawasaki disease and Guillain-Barre syndrome. Control trials have also shown clinical efficacy of IGIV in immune neutropenia, myasthenia gravis, multifocal motor neuropathy, chronic inflammatory demyelinating polyneuropathy, relapsing-remitting multiple sclerosis, myastesia gravis and refractory dermatomyastosis (Dalakas et al. Ann. Int. Med., 1997, 126721-730). IGIV has also been used to treat 50-60 unapproved conditions with beneficial effects in most of them including women with recurrent abortions of unknown causes (Carp et al., Am. J. Reprod. Immunol., 1996, 35:360-362), heparin induced thrombocytopenia (Winder et al., J. Clin. Immunol., 1998, 18:330-334), systemic vasculitis (Levy et al., Int. Arch Allergy Immunol., 1999, 119:231-238) and systemic sclerosis (Levy et al., Clin. Rhematol., 2000, 19:200). In addition, a few animal models have shown the beneficial effect of IGIV in both prevention and treatment of experimental anti-phospholipid syndrome (APS) and systemic lupes erythrematosys (SLE) (Krause et al., J. Rhematol. 1995, 22:1068-1074), which was also confirmed in humans (lupus) (Levy et al., Lupus 8: 705-712, 1999).

### SUMMARY OF THE INVENTION

Passive immunotherapy using fibril-reactive mAbs has been shown experimentally to reduce amyloid formation and also accelerate amyloidolysis. The present invention shows that human sera, as well as various sources of pooled human IgG, including pharmacologic formulations of immune globulin intravenous (IGIV), contain antibodies that specifically recognize fibrils formed from light chains (LC) and other amyloidogenic precursor proteins, including serum amyloid A (SAA), transthyretin (TTR), islet amyloid polypeptide (IAPP), and amyloid β 1-40 peptide (AD), but notably, do not react with these molecules in their native non-fibrillar forms. After isolation of the antibodies from IGIV via fibril-conjugated affinity column chromatography, the EC50 binding value for LC and Aβ fibrils was ˜15 nM—a magnitude ˜200- and 70-times less than that of the unbound fraction and unfractionated product, respectively. Comparable reactivity was found in the case of those formed from SAA, TTR, and IAPP. The purified antibodies immunostained human amyloid tissue deposits and additionally, could inhibit fibrillogenesis, as shown in fibril formation and extension assays. Most importantly, in vivo reactivity was evidenced in a murine model when the enriched antibodies were used to image amyloid, as well as expedite its removal. These promising experimental results suggest that fibril affinity-purified IGIV has potential as a diagnostic and therapeutic agent for patients with amyloid-associated disease.

The present invention provides compositions comprising IGIV or IVIG enriched for fibril binding for treating diseases or conditions associated with amyloid deposition. The IGIV may be from any animal source that has IGIV. In one embodiment, the IGIV is isolated from a human source. The present invention also provides monoclonal antibodies, polyclonal antibodies, chimeric antibodies, humanized antibodies, or single chain antibodies that mimic fibril-reactive IGIVs.

The present invention provides a method of enriching a sample of IGIV to contain a higher amount of IGIV that recognizes amyloid fibrils as compared to the starting sample of IGIV or other commercially available source of IGIV. The method of the present invention employs affinity purification.

In one embodiment, a therapeutically effective amount of IGIV or fragment thereof is administered to a subject in need thereof. The IGIV or fragment binds to the amyloid fibril thereby inhibiting or preventing formations of amyloid deposits and removing the amyloid deposit from the subject which at a minimum reduces the size of the amyloidomas in the subject.

In another embodiment, the present invention provides a method of treating or preventing amyloid formation. The present invention also provides a method of removing amyloid deposits or reducing the size of amyloidomas. Moreover, the present invention also provides a method of inhibiting or preventing and modulating the formation of amyloid deposits.

The binding of IGIV or fragment thereof to amyloid fibrils and partially denatured amyloidogenic precursor polypeptides also enables the use of IGIV as diagnostic tools. In one embodiment, the present invention provides a method of detecting amyloid deposits in a subject or in a biological sample. In another embodiment, the present invention provides a method of imaging amyloid deposits in a subject or in a biological sample. The present invention is useful for monitoring and diagnosing amyloid deposition in a subject or a sample. For use as a diagnostic tool, the IGIV or fragment thereof of the present invention may be labeled, for example, with radionuclides, contrasting agents, enzymes and dyes. The IGIV may be imaged, for example, by SPECT, CT, PET, x-ray, MRI, optical or infrared imaging and ultrasound.

### BRIEF DESCRIPTION OF THE DRAWINGS

FIG. 1 shows a graph depicting fibril binding reactivities of human sera and immune globulin contain IgG polyspecific fibril-reactive antibodies. Europium (Eu³⁺)-linked immunoassay (EuLISA) in which 400 ng of LC, AA, TTR, IAPP, or Aβ fibrils immobilized on microplate wells were exposed first to serial dilutions of (left) serum from 2 healthy adults ( [symbol] ) or (right) fibril affinity-purified (), unfractionated (□), and unbound (Δ) IgG and then to biotinylated anti-human γ-chain specific antibody. After addition of Eu³⁺-streptavidin conjugate followed by a releasing-enhancement solution, the amount of bound IgG was quantitated by time-resolved fluorescence. The values shown represent the mean±SD of triplicate analyses.

FIG. 2 shows a graph depicting fibril-binding antibodies in human immune globulin (IGIV) products by comparison of the titration curves obtained from the binding of Gammagard S/D®, Panglobulin®, and Polygam® S/D to LC fibrils immobilized on microtiter plate wells.

FIG. 3 shows a graph depicting fibril-binding antibodies in human immune globulin (IGIV) products by comparison of the titration curves of fibril affinity-purified (enriched) Gammagard S/D®, Polygam® S/D, and Panglobulin® antibodies binding to LC fibrils immobilized on microtiter plate wells.

FIG. 4 shows a graph depicting fibril-binding antibodies in a single-source of human immune globulin (IGIV) by comparison of the titration curves obtained from the binding of affinity-purified (enriched) antibodies isolated from 4 different lots of Polygam® S/D to LC fibrils immobilized on microtiter plates.

FIGS. 5A-5C show a graph depicting specificity of IGIV fibril-binding antibodies by comparison of the capability of native (non-fibrillar) amyloidogenic precursor proteins vs. their fibrillar counterparts to inhibit, in a dose dependent fashion, the binding of fibril affinity-purified (enriched) IGIV to (A) LC, (B) TTR, or (C) Aβ fibrils immobilized on microtiter plates.

FIG. 6 shows a graph showing that IGIV fibril-reactive antibodies recognize partially denatured, non-fibrillar amyloidogenic proteins by comparison of the titration curves obtained from the binding of affinity-purified IGIV antibodies to LC or TTR proteins vs. their fibrillar counterparts, each immobilized (dried) on microtiter plate wells. The data on LC proteins were scaled down by a factor of 4 to account for differences in epitope density.

FIG. 7 shows a graph depicting inhibition of LC fibrillogenesis by human immune globulin (IGIV) and effect of fibril affinity-purified (enriched) and unfractionated IGIV on the growth of LC fibrils (as measured by ThT fluorescence in the microtiter plate assay).

FIG. 8 shows a graph depicting inhibition of Aβ fibrillogenesis by human immune globulin (IGIV) and effect of fibril affinity-purified (enriched) and unfractionated IGIV on the growth of Aβ fibrils (as measured by ThT fluorescence in the microtiter plate assay).

FIG. 9 shows a graph depicting inhibition of IAPP fibrillogenesis by human immune globulin (IGIV), (Upper) kinetic traces of IAPP fibril formation in the presence or absence of enriched IGIV, as measured by ThT fluorescence in the microtiter plate assay, (Lower) comparison of the dose-dependent inhibitory effect of IGIV on IAPP fibrillogenesis, as determined from reaction end points. The dashed line represents the fluorescence intensity of less ordered IAPP intermediates.

FIGS. 10A-10D show an image depicting imaging of amyloid by radioiodinated fibril-reactive IGIV antibodies. Reconstructed, 3-dimensional, micro-CT and co-registered micro-SPECT (pseudocolored red)/micro-CT images of a BALB/c mouse bearing a 50 mg s.c. AL amyloidoma (arrow) and obtained 72 hrs post i.v. injection of 15 mg of 125I-labeled fibril affinity-purified (enriched) IGIV (specific activity, ˜10 mCi/mg, 0.4 MBq/mg). (A and C) Micro-CT images (sagital and axial views, respectively). (B and D) Co-registered micro-SPECT/micro-CT images (sagital and axial views, respectively).

FIG. 11 shows a graph depicting biodistribution of radioiodinated fibril-reactive human immune globulin (IGIV) by comparison of tissue vs. amyloidoma uptake 72 hrs after injection of 125I-labeled fibril affinity-purified (enriched) IGIV into mice with (1 and 2) or without (Cont 1, 2 and 3) induced human AL or ATTR amyloidomas.

FIG. 12 shows a graph depicting enhancement of serum anti-fibril reactivity by comparison of the titration curves obtained from the binding of LC fibrils immobilized on microtiter plate wells to sera from patients with ALk, ALl, or ATTR amyloidosis before and after addition of 2 mg/ml fibril affinity-purified (enriched) IGIV.

FIG. 13 shows a graph depicting enhancement of serum reactivity to autologous amyloid extracts by addition of anti-fibril enriched IGIV by comparison of the titration curves obtained from the binding of homologous amyloid extracts immobilized on microtiter plate wells to sera from patients with ALk and ALl amyloidosis with and without 2 mg/ml of fibril affinity-purified (enriched) or native (unfractionated) IGIV.

FIG. 14 shows a graph depicting pharmokinetics of human immune globulin (IGIV) infusions and reactivity of LC-related (rV16 Jto) fibrils immobilized on microtiter plate wells to sera from an ALl patient who received IGIV (Gammagard S/D®) infusions over a >1 year time period. Amount infused: Day 1: 15 g; Days 2, 6, and 13: 25 g. No infusions were given for ˜7 weeks, after which they were resumed on a weekly basis at a dose of 30 gm.

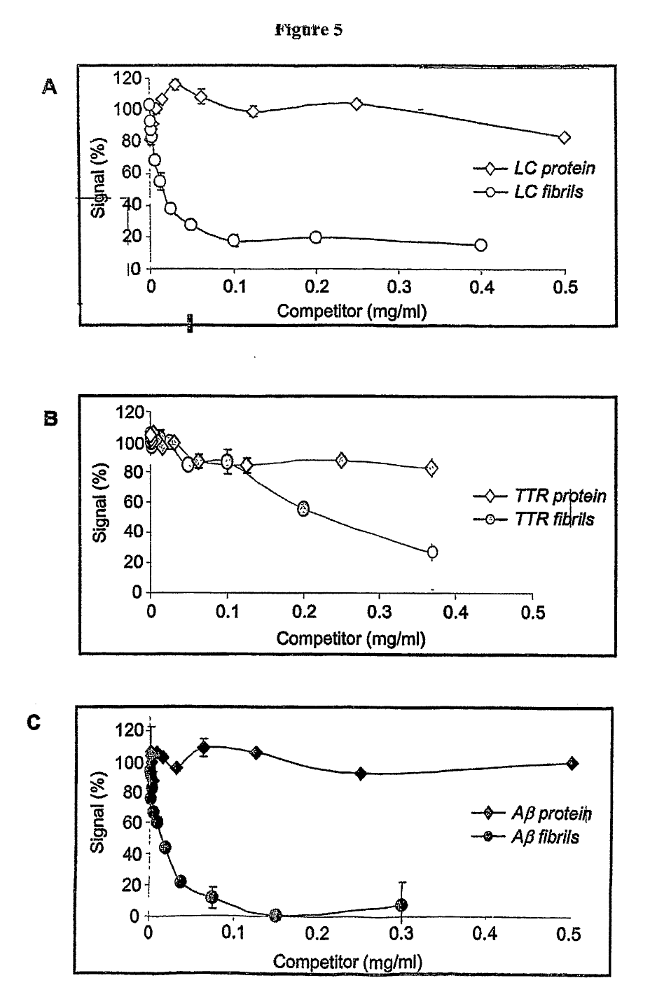

FIGS. 5A-15C show a graph depicting the specificity of affinity-purified IgG antibodies for fibrils. Comparison of the capability of native (non-fibrillar) amyloidogenic precursor proteins or peptides () vs. their fibrillar counterparts (⋄) to inhibit, in a dose-dependent fashion, the binding of fibril affinity-purified (enriched) IgG to (A) LC- and (B) Aβ-related fibrils immobilized on microtiter plate wells. (C) Comparison of the capability of soluble Aβ vs. Aβ, LC, TTR, and IAPP fibrils (protein concentration, 0.3-0.4 mg/ml) co-incubated with 80 nM of affinity-purified (enriched) IgG to inhibit antibody binding to Aβ fibrils immobilized on microtiter plate wells (400 ng/well).

FIGS. 16A-16H show a graph and image depicting the inhibition of fibrillogenesis by IgG fibril-reactive antibodies. Fibril formation: Affinity-purified, residual, or native IgG was incubated at 37° C. with either LC (5 μM), Aβ (90 μM), or IAPP (50 μM) and the extent of fibril formation measured by ThT fluorescence. (A) Dose-dependent effect of enriched (▪), residual ( [symbol] ), and unfractionated (□) antibodies on LC fibrillogenesis at 18 h. (B) Time-dependent effect of enriched (▪), residual ( [symbol] ) and unfractionated (□) IgG (1 μM) on Aβ fibril formation (no antibody [□]). (C) Kinetic traces of IAPP fibril formation in the presence or absence of enriched, residual, or unfractionated IgG (1 □M) as measured by ThT fluorescence. (D) Comparison of the dose-dependent inhibitory effect of enriched (▪), residual ( [symbol] ), and unfractionated (□) IgG (1 μM) on IAPP fibrillogenesis, as determined from reaction end points. The dashed line represents the fluorescence intensity of less-ordered intermediates formed in the initial phase of IAPP fibrillogenesis (16). (E) Electron micrographs of 50 μM IAPP alone and in the presence of 1 μM unfractionated or enriched IgG (uranyl acetate stain; original magnification, ×50,000; scale: bar, 200 nm). Fibril extension: Dose-dependent effect of enriched (), residual ( [symbol] ), and unfractionated (□) IgG on recruitment of amyloidogenic precursors (biotinyl-LC [250 nM], biotinyl-Aβ [50 nM], and IAPP [28 μM]) onto pre-formed, sonicated (F) LC, (G) Aβ fibrils, or (H) IAPP (400 ng/well) immobilized on microtiter plate wells. LC and Aβ fibril elongation was monitored by Eu³⁺-time-resolved fluorescence and that of IAPP by ThT fluorescence.

FIG. 17 shows an image depicting an immunostain of human amyloid tissue deposits by fibril affinity-purified (enriched) IgG antibodies. Left panels: Congo red-stained sections (viewed by polarizing microscopy) of kidney, pericardium, ovary, myocardium, and pancreas obtained from patients with ALκ, ALλ, AA, ATTR, and AIAPP amyloidosis, respectively, and cerebral cortical tissue from an individual with Alzheimer's disease. Middle and Right panels: Immunoperoxidase stains. Primary reagents; enriched IgG before and after fibril absorption, respectively; secondary reagent, biotinylated goat anti-human IgG. Original magnification: kidney ×200; pericardium, ×400; ovary, ×200; myocardium, ×80; and pancreas, ×200, brain, ×400.

FIG. 18 shows an image depicting the amyloidolytic activity of fibril-reactive IgG antibodies. SCID mice bearing 100-mg ALκ, ALλ or ATTR s.c. amyloidomas received (in 2 divided sites), on days 0, 2, 4, 6, 8, and 10, s.c. 0.25 ml injections containing 0.25 mg of fibril affinity-purified (enriched) or unfractionated IGIV in PBS, as well as PBS alone. Amyloidomas (enclosed within the red boxes) were excised from euthanized animals 18 days after the last injection (weights of amyloidomas are as indicated). Also illustrated is the extent of cellular infiltration, as seen in H&E-stained sections of amyloidomas (original magnification, ×200).

### DETAILED DESCRIPTION

### A. General Description

The present invention is based, in part, on the discovery that pharmacologic sources of IGIV contain IgG antibodies that specifically recognize a generic conformational epitope(s) expressed by fibrils formed from at least 5 different types of amyloidogenic precursors, i.e., LC, SAA, TTR, IAPP, and Aβ (and partially denatured LC and TTR molecules), but notably do not when they are in their soluble native states. In this respect, these antibodies differ from, for example, the anti-Aβ reactive components found in human sera (Fang et al., 1995; Hyman et al., 2001; Weksler et al., 2002; Du et al., 2001), an IGIV product (Dodel et al., 2002), those induced by Aβ immunization of mice (Solomon et al., 1996; 1997; Bard et al., 2000, 2003; DeMattos et al., 2001; Dodart et al., 2002; Morley et al., 2002; Du et al., 2003; Wilcock et al., 2004; Bussière et al., 2004; Brendza et al., 2005; Horikoshi et al., 2004), or the IgM mAbs generated from peripheral blood lymphocytes (Geylis et al., 2005), none of which have been shown to differentiate between the soluble and fibrillar configurations or to bind heterologous fibrils.

Although fibril-reactive antibodies represent ˜0.2% of the IgG content of IGIV, it is possible, through affinity chromatography, to isolate the active fraction and document its binding specificity by a sensitive fluoroimmunoassay, as well as immunohistochemically, where it is shown to immunostain amyloid deposits in tissue. The present invention is based on the finding that IGIV enriched for fibril binding by affinity chromatography binds amyloid deposits in tissue. Further, these molecules could inhibit LC, TTR, IAPP, and Aβ fibrillogenesis, as evidenced in fibril formation and extension assays. In other studies, it was demonstrated in an in vivo experimental murine model that the purified antibodies, when radiolabeled, could serve as diagnostic reagents capable of imaging amyloid deposits. Moreover, therapeutic activity was documented by their acceleration of amyloidolysis in animals bearing human AL or ATTR amyloidomas.

### B. Definitions

As used herein, a “diagnostic agent” or “imaging agent” refers to agents including those that are pharmaceutically acceptable agents that can be used to localize or visualize amyloid deposits by various methods.

As used herein, “gamma globulin” is the serum globulin fraction that is mainly composed of IgG molecules.

As used herein, “IGIV” or “intravenous immunoglobulins” refers to gamma globulin preparations suitable for intravenous use, such as those IGIV preparations commercially available from several sources.

As used herein, “fragments” of IGIV or gamma globulin are portions of intact immunoglobulins such as Fc, Fab, Fab′, F(ab′)₂ and single chain immunoglobulins.

As used herein, the phrase “specifically (or selectively) binds to” or “specifically (or selectively) immunoreactive with” refers to a binding reaction which is determinative of the presence of the molecule of interest in the presence of a heterogeneous population of proteins and other biologics. Thus, under designated assay conditions, the specified ligands (e.g., an antibody) bind to a particular molecule (e.g., an epitope on amyloid fibril) and do not bind in a significant amount to other molecules present in the sample. In affinity purification, the ligand may be the amyloid fibril conjugated to an affinity purification matrix and the molecule of interest is the IGIV being enriched for amyloid fibril binding.

As used herein, “pharmaceutical composition” or “formulation” refers to a composition comprising an agent or compound together with a pharmaceutically acceptable carrier or diluent. A pharmaceutically acceptable carrier includes, but is not limited to, physiological saline, ringers, phosphate buffered saline, and other carriers known in the art. Pharmaceutical compositions may also include stabilizers, anti-oxidants, colorants, and diluents. Pharmaceutically acceptable carriers and additives are chosen such that side effects from the pharmaceutical agent are minimized and the performance of the agent is not canceled or inhibited to such an extent that treatment is ineffective.

As used herein, “subject” can be a human, a mammal, or an animal. The subject being treated is a patient in need of treatment.

As used herein, “therapeutically effective amount” refers to that amount of the agent or compound which, when administered to a subject in need thereof, is sufficient to effect treatment. The amount of IGIV which constitutes a “therapeutically effective amount” will vary depending on the severity of the condition or disease, and the age and body weight of the subject to be treated, but can be determined routinely by one of ordinary skill in the art having regard to his/her own knowledge and to this disclosure.

### C. Specific Embodiments

IGIV

IGIV are gammaglobulins isolated from the blood of donors and are suitable for intravenous administration. IGIV can be isolated from different mammals, including non-human sources, such as mouse, rat, hamster, guinea pig, dog, cat, rabbit, pig, goat, sheep, cow, chimpanzee, and monkey. In one embodiment of the invention, human IGIV preparations are used for intravenous administration. Human IGIV preparations are available from various commercial sources. The commercially available IGIV preparations contain mainly IgG molecules. IGIV has been used in replacement therapy in primary immunodeficiency syndromes and in secondary immunodeficiencies as well as for the prevention and treatment of infectious diseases. Furthermore, IGIV has also been used for immune modulation of patients with autoimmune and immune-complex diseases. See, Martha M. Eibl, “Intravenous Immunoglobulin: A Review”, Immunodeficiency Reviews, 1 (Suppl.), pp 1-42 (1989).

The present invention provides IGIV as a whole molecule or fragments thereof such as the F(ab′)₂ or Fc fragment by itself in treating subjects. Prior to administration, the IGIV preparation may be subject to treatment such as enzymatic digestion (e.g. with pepsin, papain, plasmin, glycosidases, nucleases, etc.), heating, etc. and/or further fractionated but will normally be used as commercially available. Thus, administered compositions may comprise primarily intact antibody, antibody fragments, or mixtures thereof. Hence, by IGIV fragments is meant preparations of immunoglobulin fragments suitable for human intravenous administration. In one embodiment, the IGIV or fragments thereof are enriched for binding to amyloid fibrils and to partially denatured amyloidogenic precursor polypeptides. The IGIV or fragments thereof are effective for treating subjects suffering from amyloid deposits. They can be used to remove amyloid deposits or inhibit or modulate the formation of amyloid deposits from a patient. They can be used to detect amyloid fibrils in subjects.

The present invention also provides monoclonal and polyclonal antibodies that mimic fibril-reactive IGIVs. These antibodies will bind to amyloid fibrils and partially denatured amyloidgenic precursor polypeptides. They can be used to treat subjects suffering from amyloid deposits. They are useful for removing and inhibiting or modulating the formation of amyloid deposits from a patient. Monoclonal and polyclonal antibodies of the present invention can be obtained by immunizing animals with peptides or other molecules that mimic the IGIV fibril epitopes. These antibodies will bind IGIV fibril epitopes on amyloid fibrils and partially denatured amyloidogenic precursor proteins.

Polyclonal antibodies that mimic fibril-reactive IGIVs can be prepared by any methods known in the art. As described, polyclonal antibodies may be prepared by immunizing a suitable subject with polypeptides, peptides or molecules that mimic the IGIV fibril epitopes. The desired polyclonal antibodies may be isolated from the sera of the subject. In one embodiment, the polyclonal antibody compositions are ones that have been selected for antibodies that recognize or bind specifically to amyloid fibrils or partially denatured amyloidogenic precursor proteins or polypeptides.

Monoclonal antibodies that mimic fibril-reactive IGIVs may be made by the hybridoma method first described by Kohler et al, Nature, 256:495 (1975), or may be made by recombinant DNA methods (see, e.g., U.S. Pat. No. 4,816,567, which is herein incorporated by reference in its entirety). Monoclonal antibodies may also be isolated from phage antibody libraries using the techniques described in Clackson et al., Nature, 352:624-628 (1991) and Marks et al., J. Mol. Biol., 222:581-597 (1991), for example.

The phrase “specifically (or selectively) binds” to an antibody or “specifically (or selectively) immunoreactive with,” refers to a binding reaction that is determinative of the presence of the amyloid fibrils or partially denatured amyloidogenic precursor polypeptides in a heterogeneous population of proteins and other biologics. Thus, under designated immunoassay conditions, the specified antibodies bind partially denatured amyloidogenic precursor proteins or amyloid fibrils at least two times the background and do not substantially bind in a significant amount to other proteins or biologics present in the sample. A variety of immunoassay formats may be used to select antibodies specifically immunoreactive with a partially denatured amyloidogenic precursor proteins. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The monoclonal antibodies of the present invention also include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)), such as binding to amyloid fibrils and to partially denatured amyloidogenic precursor proteins. A chimeric antibody is a molecule in which different portions are derived from different animal species, such as those having a variable region derived from a murine mAb and a human immunoglobulin constant region. Chimeric antibodies may be obtained by splicing the genes from a mouse antibody molecule of appropriate antigen specificity together with genes from a human antibody molecule of appropriate biological activity can be used (Morrison et al., 1984, Proc. Natl. Acad. Sci. USA, 81:6851 5; Neuberger et al., 1984, Nature, 312:604 8; Takeda et al., 1985, Nature, 314:452 4).

The present invention also includes humanized antibodies (see, e.g., U.S. Pat. No. 5,585,089 which is incorporated by reference in its entirety) that bind amyloid fibrils and partially denatured amyloidogenic precursor proteins. “Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Moreover, the present invention includes single chain antibodies (U.S. Pat. No. 4,946,778; Bird, 1988, Science 242:423 6; Huston et al., 1988, Proc. Natl. Acad. Sci. USA 85:5879 83; and Ward et al., 1989, Nature 334:544 6) that bind amyloid fibrils and partially denatured amyloidogenic precursor proteins. Single chain antibodies are formed by linking the heavy and light chain fragments of the Fv region via an amino acid bridge, resulting in a single chain polypeptide.

Enrichment of IVIG for Amyloid Fibril Binding

The present invention provides IGIV that binds to amyloid fibrils and to partially denatured amyloidogenic precursor polypeptides. The inventors demonstrated that commercially available IGIV contain IgG antibodies that bind amyloid fibrils using highly sensitive fluoroimmunoassay. The IgG antibodies recognized a conformational epitope(s) expressed on LC, AA, TTR, IAPP, and Aβ amyloid fibrils, and on partially denatured LC and TTR proteins. However, these IgG antibodies did not bind these molecules in their native non-fibrillar states.

Generally, a sample of commercially available IGIV contains only a small amount (˜0.2%) fibril reactive antibodies. The inventors have found that a sample of commercially available IGIV may be enriched for amyloid fibril binding using an amyloid fibril conjugated affinity column. The IGIV isolated from the amyloid fibril affinity column is enriched for binding amyloid fibril as compared to or relative to the starting material. The present invention provides IGIV or fragments thereof enriched for fibril binding. Such enrichment may comprise about a 10%, 20%, 50%, 75%, 100%, 200%, 400% or more increase in binding compared to the starting material. In another embodiment, such enrichment may comprise about a 2-fold, 3-fold, 4-fold, 5-fold, 7-fold, 10-fold, 20-fold, 50-fold, 100-fold, 500-fold or more binding compared to the starting material. In still another embodiment, the purified fraction may comprise about 1%, 5%, 10%, 25%, 50%, 75%, 80% or more fibril reactive antibodies. IGIV enriched or concentrated for amyloid fibril binding may be obtained by various affinity purification methods.

Affinity purification (also called affinity chromatography) makes use of specific binding interactions between molecules. Affinity purification broadly refers to separation methods based on a relatively high binding capacity (“affinity”) of a target material to be purified, generally termed a “ligate”, for a complementary ligand. Affinity purifications can be accomplished in solution. However, more typically, a particular ligand is chemically immobilized or “coupled” to a solid support so that when a complex mixture is passed over the column, only those molecules having specific binding affinity to the ligand are purified. In the affinity purification method of the present invention, the ligand used for isolating IGIV enriched for fibril binding is amyloid fibrils. The amyloid fibrils may be from naturally occurring sources, by recombinant means, or from synthetic sources. See Table 1 for a list of amyloid fibrils that may be used in these methods and PCT/US99/11200 and PCT/US 01/11043 for methods of creating synthetic amyloid fibrils. Affinity purification generally involves the following steps:
1. Incubate crude sample with the immobilized ligand support material to allow the target molecule in the sample to bind to the immobilized ligand.
2. Wash away non-bound sample components from solid support.
3. Elute (dissociate and recover) the target molecule from the immobilized ligand by altering the buffer conditions so that the binding interaction no longer occurs.
   A single pass of a sample through an affinity column can achieve greater than 1,000 fold purification of a molecule from a crude mixture.

Affinity purification involves the separation of molecules in solution (mobile phase) based on differences in binding interaction with a ligand that is immobilized to a stationary material (solid phase). A support or matrix in affinity purification is any material to which a biospecific ligand may be covalently attached. Typically, the material to be used as an affinity matrix or resin is insoluble in the system in which the target molecule is found. Usually, but not always, the insoluble matrix is a solid. Hundreds of substances have been described and employed as affinity matrices.

Useful affinity supports are those that contain: a high surface area to volume ratio, chemical groups that are easily modified for covalent attachment of ligands, minimal nonspecific binding properties, good flow characteristics and mechanical and chemical stability. Ideally, matrices for ligand immobilization should have a large surface area and comprise an open and loose porous network to maximize interaction of matrix-bound ligand with ligate (molecule of interest during the separation procedure). The matrix should be chemically and biologically inert, at the very least toward the ligand and ligate; be adapted for ligand immobilization; and be stable under reaction conditions employed, for example during matrix activation, ligand binding, and ligand-ligate complex formation, especially with respect to the solvent, pH, salt, and temperature employed. The matrix should also be stable for a reasonable length of time under ordinary storage conditions. To minimize competition for the target material and maximize purity of recovered product, supports for immobilization of ligands, especially biospecific ligands, should be free from extraneous ion exchange sites, and should not promote non-specific binding. Matrices, especially those used in pressurized affinity separation techniques, should be mechanically strong and be able to withstand at least the moderate pressures typical of these conventional systems (up to about 5 bar, for example). Matrices may be derivatized, for example, to promote ligand immobilization or to permit improved ligand/target interaction.

There are a number of useful matrix materials such as agarose gels; cellulose; dextran; polyacrylamide; hydroxyalkylmethacrylate gels; polyacrylamide/agarose gels; ethylene copolymers, especially with polyvinyl acetate; copolymers of methacrylamide, methylene bis-methacrylamide, glycidyl-methacrylate and/or allyl-glycidyl-ether (such as Eupergit C, Rohm Pharma, Darmstadt, West Germany); and diol-bonded silica. The present invention provides fibrils linked covalently to an N-hydroxysuccinimide (NHS)-activated Sepharose® 4 fast-flow pre-activated agarose matrix.

Most commonly, ligands are immobilized or “coupled” directly to solid support material by formation of covalent chemical bonds between particular functional groups on the ligand (e.g., primary amines, sulfhydryls, carboxylic acids, aldehydes) and reactive groups on the support (see later discussions in this section). However, other coupling approaches are also possible.

Most affinity purification procedures involving protein-ligand interactions use binding buffers at physiologic pH and ionic strength, such as phosphate buffered saline (PBS). For obvious reasons, this is especially true when antibody-antigen or native protein-protein interactions are the basis for the affinity purification. Once the binding interaction occurs, the support is washed with additional buffer to remove unbound components of the sample. Nonspecific (e.g., simple ionic) binding interactions can be minimized by adding low levels of detergent or by moderate adjustments to salt concentration in the binding and/or wash buffer. Finally, elution buffer is added to break the binding interaction and release the target molecule, which is then collected in its purified form. Elution buffer can dissociate binding partners by extremes of pH (low or high), high salt (ionic strength), the use of detergents or chaotropic agents that denature one or both of the molecules, removal of a binding factor or competition with a counter ligand. In most cases, subsequent dialysis or desalting is required to exchange the purified protein from elution buffer into a more suitable buffer for storage or downstream analysis.

The most widely used elution buffer for affinity purification of proteins is about 0.1 M glycine•HCl, at about pH 2.5-3.0. This buffer effectively dissociates most protein-protein and antibody-antigen binding interactions without permanently affecting protein structure. However, some antibodies and proteins are damaged by low pH, so eluted protein fractions should be neutralized immediately by collecting the eluting fractions in tubes containing 1/10th volume of alkaline buffer such as about 1 M Tris.HCl, at about pH 8.5 to 9.0. Other elution buffers for affinity purification of proteins are well known to one of ordinary skill in the art.

Affinity purification may also be carried out in batch mode, for example in a beaker or a similar container. The ligand, amyloid fibrils or partially denatured amyloidogenic precursor polypeptides may be conjugated to an appropriate resin and placed in a beaker for affinity purification. A sample of IGIV may be poured into the beaker and swirled around to allow binding to the amyloid fibrils or partially denatured amyloidogenic precursor polypeptides and washed in the beaker with buffers. IGIV that binds amyloid fibrils may be eluted and isolated as described earlier.

Uses of IGIV Compositions

The present invention provides IGIV compositions and IGIV compositions enriched for binding to amyloid fibrils and to partially denatured amyloidogenic precursor polypeptides for treating diseases and conditions associated with amyloid deposition. The IGIV binds amyloid deposits, inhibiting and modulating the formation of amyloid deposition. Moreover, the binding of the IGIV to amyloid deposits leads to reducing the size of the amyloidomas and removal or resolution of the amyloidomas in a patient.

In one embodiment of the invention, the present invention provides a method of treating a subject having amyloid deposition comprising administering to the subject a therapeutically effective amount of intravenous immune globulin (IGIV) or fragment thereof, wherein the IGIV or fragment thereof binds an amyloid fibril.

In another embodiment, the present invention provides a method of removing amyloid deposits from a subject comprising administering to the subject an effective amount of intravenous immune globulin (IGIV) or fragment thereof to remove amyloid deposits, and allowing the IGIV or fragment thereof to bind amyloid deposits, thereby removing the amyloid deposit from the subject.

Moreover, the present invention provides a method of inhibiting the formation of amyloid deposits in a subject comprising administering to the subject an effective amount of intravenous immune globulin (IGIV) or fragment thereof to inhibit formation of amyloid deposits, and allowing the IGIV or fragment thereof to bind amyloid-forming precursor protein, thereby inhibiting the formation of amyloid deposits.

Further, the present invention provides a method of modulating the formation of amyloid deposits in a subject comprising administering to the subject an effective amount of intravenous immunoglobulin (IGIV) or fragment thereof to modulate formation of amyloid deposits, and allowing the IGIV or fragment thereof to bind an amyloid fibril, thereby modulating formation of amyloid deposits.

As an alternate embodiment, the present invention also provides IGIV and amyloid fibril binding enriched IGIV for diagnostic methods. The present invention provides a method of detecting amyloid deposits in a subject comprising administering to the subject an effective amount of intravenous immunoglobulin (IGIV) or fragment thereof to detect amyloid deposits and allowing the IGIV or fragment thereof to bind amyloid deposits, and detecting amyloid deposits.

The present invention also provides a method of imaging amyloid deposits in a subject comprising administering to the subject an effective amount of intravenous immunoglobulin (IGIV) or fragment thereof to image amyloid deposits and allowing the IGIV or fragment thereof to bind amyloid deposits, and obtaining an image of the amyloid deposits.

Pharmaceutical Compositions of IGIV

The present invention provides pharmaceutical composition or formulations comprising therapeutically effective amount of IGIV for the treatment of amyloidosis in a subject or patient. The compositions could be used to remove, inhibit, detect, image and modulate the formation of amyloid deposits in a subject. The IGIV compositions of the present invention may be enriched for fibril binding and or binding to partially denatured amyloidogenic precursor polypeptides.

The IGIV preparations used according to the present invention may include commercially available preparations of intact IGIV and preparations of the fragments of IGIV. The IGIV or fragments thereof may be enriched for binding to amyloid fibrils and to partially denatured amyloidogenic precursor polypeptides. Recombinantly produced gamma globulins and their fragments may also be used according to this invention. The use of recombinant single chain antibodies is also envisioned.

The dosage of IGIV and the method of administration will vary with the severity and nature of the particular condition being treated, the duration of treatment, the adjunct therapy used, the age and physical condition of the subject of treatment and like factors within the specific knowledge and expertise of the treating physician. However, single dosages for intravenous and intracavitary administration can typically range from 400 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 400 mg/kg/day for 5 consecutive days per month or 2 g/kg/day once a month. The IGIV enriched for fibril binding of the present invention was found to be effective in reducing the size of amyloidomas in mice induced to develop amyloidomas.

In another embodiment of this invention, the IGIV preparation is administered via the subcutaneous route. The typical dosage for subcutaneous administration can range from 4 mg to 20 mg per kg body weight. The IGIV according to the present invention was found to be effective in inhibiting metastasis in mice when administered subcutaneously in the dose 200 μg/mouse.

According to the present invention IGIV may be administered as a pharmaceutical composition containing a pharmaceutically acceptable carrier. The carrier must be physiologically tolerable and must be compatible with the active ingredient. Suitable carriers include sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

Detecting and Imaging Amyloid Deposits

The present invention further provides a method of detecting and imaging amyloid deposits using IGIV. The method of this invention determines the presence and location of amyloid deposits in an organ or body area, for example the brain, of a subject. The present method comprises administration of a detectable quantity or an imaging effective quantity of IGIV, to a subject or patient. A “detectable quantity” means that the amount of the detectable compound that is administered is sufficient to enable detection of binding of the compound to amyloid. An “imaging effective quantity” means that the amount of the detectable compound that is administered is sufficient to enable imaging of binding of the compound to amyloid.

IGIV may be tagged with an diagnostic or imaging agent known in the art, such as radionuclides, enzymes, dyes, fluorescent dyes, gold particles, iron oxide particles and other contrast agents including paramagnetic molecules, x-ray attenuating compounds (for CT and x-ray) contrast agents for ultrasound. Appropriate agents for imaging amyloid deposits include iron oxide particles, dyes, fluorescent dyes, NMR labels, scintigraphic labels, gold particles, PET labels, ultrasound contrast media, and CT contrast media. A variety of different types of substances can serve as the reporter group for tagging IGIV, including but not limited to enzymes, dyes, radioactive metal and non-metal isotopes, fluorogenic compounds, fluorescent compounds, etc.

Methods for preparation of antibody conjugates of the antibodies (or fragments thereof) of the invention useful for detection, monitoring are described in U.S. Pat. Nos. 4,671,958; 4,741,900 and 4,867,973, the contents of which are hereby incorporated by reference. Also known in the art is the method of using monoclonal antibodies as probes for imaging of Aβ (Majocha et al., J. Nucl. Med., 33: 2184 (1992); Majocha et al., WO 89/06242 and Majocha et al., U.S. Pat. No. 5,231,000).

The invention employs tagged IGIV which, in conjunction with non-invasive neuroimaging techniques such as magnetic resonance spectroscopy (MRS) or imaging (MRI), or gamma imaging such as positron emission tomography (PET) or single-photon emission computed tomography (SPECT), or CT, x-ray, optical or infrared imaging, and ultrasound, are used to quantify amyloid deposition in vivo. The term “in vivo imaging” refers to any method which permits the detection of labeled IGIV.

For purposes of in vivo imaging, the type of detection instrument available is a major factor in selecting a given label. For instance, radioactive isotopes such as ¹²⁵I are particularly suitable for in vivo imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using nuclear imaging wherein emitted radiation of the appropriate energy is detected. Methods of nuclear imaging include, but are not limited to, SPECT and PET. Preferably, for SPECT detection, the chosen radiolabel will lack a particulate emission, but will produce a large number of photons in a 140-200 keV range. For PET detection, the radiolabel will be a positron-emitting radionuclide such as ¹⁹F which will annihilate to form two 511 keV gamma rays which will be detected by the PET camera.

The methods of the present invention may use isotopes detectable by nuclear magnetic resonance spectroscopy for purposes of in vivo imaging and spectroscopy. Elements particularly useful in magnetic resonance spectroscopy include ¹⁹F, Gd and ¹³C.

Suitable radioisotopes for purposes of this invention include beta-emitters, gamma-emitters, positron-emitters, and x-ray emitters. These radioisotopes include ¹³¹I, 123I, ^{99m}Tc, ¹¹¹In, ¹²⁴I, ¹⁸F, ¹¹C, ⁷⁵Br, and ⁷⁶Br. Suitable stable isotopes for use in Magnetic Resonance Imaging (MRI) or Spectroscopy (MRS), according to this invention, include ¹⁹F, Gd and ¹³C. Suitable radioisotopes for in vitro quantification of amyloid in homogenates of biopsy or post-mortem tissue include ¹²⁵I, ¹³¹I, ¹²³I, ^{99m}Tc, ¹⁴C, and ³H. The preferred radiolabels are ¹¹C, ¹²⁴I or ¹⁸F for use in PET in vivo imaging, ¹²³I, ^{99m}Tc, ¹¹¹In or ¹²⁵I for use in SPECT imaging, ¹⁹F or Gd for MRS/MRI, and ¹²⁵I, ³H or ¹⁴C for in vitro studies. However, any conventional method for visualizing diagnostic probes can be utilized in accordance with this invention.

The method may be used to diagnose AD in mild or clinically confusing cases. This technique would also allow longitudinal studies of amyloid deposition in human populations at high risk for amyloid deposition such as Down's syndrome, familial AD, and homozygotes for the apolipoprotein E4 allele (Corder et al., Science 261: 921 (1993)). A method that allows the temporal sequence of amyloid deposition to be followed can determine if deposition occurs long before dementia begins or if deposition is unrelated to dementia. This method can be used to monitor the effectiveness of therapies targeted at preventing amyloid deposition.

Generally, the dosage of the detectably labeled IGIV will vary depending on considerations such as age, condition, sex, and extent of disease in the patient, contraindications, if any, concomitant therapies and other variables, to be adjusted by a physician skilled in the art.

Administration to the subject may be local or systemic and accomplished intravenously, intraarterially, intrathecally (via the spinal fluid) or the like. Administration may also be intradermal or intracavitary, depending upon the body site under examination. After a sufficient time has elapsed for IGIV to bind with the amyloid, for example 30 minutes to 48 hours, the area of the subject under investigation is examined by routine imaging techniques such as MRS/MRI, SPECT, planar scintillation imaging, PET, and any emerging imaging techniques, as well. The exact protocol will necessarily vary depending upon factors specific to the patient, as noted above, and depending upon the body site under examination, method of administration and type of label used; the determination of specific procedures would be routine to the skilled artisan.

Kits for Using IGIV

The present invention also provides kits for diagnosis, prognosis, monitoring, or detecting amyloidosis in a subject. The kit contains IGIV and may be means for enriching the IGIV for fibril binding. The kit may include means for affinity purification of the IGIV, such as an affinity matrix containing amyloid fibril conjugated to resin. Alternatively, the IGIV in the kit may be enriched for fibril binding.

The IGIV in the kit can be tagged with a label. Alternatively, other components can be included in the kit for tagging the IGIV. The present invention also contemplates kits comprising other components for diagnosing and monitoring the formation of amyloid deposits in a subject, and determining the prognosis of the subject. In one embodiment, the components of the kit are packaged either in aqueous medium or in a lyophilized form.

In a further embodiment, the kit may comprise a container with a label. Suitable containers include, for example, bottles, vials, and test tubes. The containers may be formed from a variety of materials such as glass or plastic. The container may comprise materials desirable from a commercial and user standpoint, including buffers, diluents, filters, needles, syringes, and package inserts with instructions for use.

The IGIV in the kit can be packaged with a container for diagnosing or detecting amyloid deposits in a patient. The kit may contain a label, such as a radioactive metal ion or a moiety for attaching to IGIV. The label can be supplied either in fully conjugated form, in the form of intermediates or as separate moieties to be conjugated by the user of the kit.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Human Sera and Immune Globulin Preparations Contain Fibril-Reactive IgG Antibodies

### Materials and Methods

Proteins and Peptides: Monoclonal serum Igs obtained from patients with multiple myeloma or AL amyloidosis were isolated and purified as previously described (Solomon et al., 1985). Human γ-globulin (Cohn Fraction II), collagen, insulin, bovine serum albumin (BSA), elastin, thyroglobulin, hen egg white ovalbumin, and calf thymus ds DNA were purchased from Sigma-Aldrich (St. Louis, Mo.) and gelatin from Bio-Rad (Hercules, Calif.). The IGIV preparations (Gammagard S/D®, Polygam® S/D, and Panglobulin®) and the human IgG subclass Profile ELISA kit were products, respectively, of Baxter Health Corp. (Westlake Village, Calif.) and Zymed Laboratories, Inc. (San Francisco, Calif.). Synthetic, i.e., recombinant, λ6 LC variable region (rV_{λ}6) components (Jto, Wil) were produced in an E. coli expression system (Wall et al., 1999). Recombinant mutant (V30M) TTR was provided by Dr. Joel N. Buxbaum. IAPP, the peptide encompassing the N-terminal 40 residues of Aβ (Aβ₁₋₄₀), and its Cys-1 analog were synthesized at the Keck Biotechnology Center (Yale University, New Haven, Conn.).

Lyophilized protein preparations were reconstituted in distilled water to obtain a 1 mg/ml solution and 10×PBS containing 0.5% sodium azide was added to a final concentration of 1× (PBSA). LCs (rV_{λ}6) were sterile-filtered and rendered aggregate-free using a 0.22 μm PVDF 25 mm Millex®-GV syringe-driven filter unit (Millipore, Billerica, Mass.). The IAPP and Aβ preparations were disaggregated by sequential exposure to trifluoroacetic acid and hexafluoroisopropanol (Murphy et al., 2001). Prior to study, all proteins or peptides were centrifuged at 14000 rpm (20800×g) for 25 min and, in the case of Aβ, 20000 rpm (50000×g) for 18 hrs, and aliquoted for immediate use or stored at 4° C. for up to 2 wks. The protein concentration of rV_{λ}6 was determined by absorbance at 280 nm using a molar extinction coefficient (E^{1%}) of 13370 and a mol wt of 12018 daltons and that of the other proteins or peptides, by the Micro BCA® Protein Assay Kit (Pierce, Rockford Ill.) using BSA as a standard.

Amyloid extraction and chemical characterization: Amyloid fibrils were extracted from tissue samples obtained post-mortem from patients with κ- or λ-LC-, AA, or TTR-associated amyloidosis (ALκ, ALλ, AA, or ATTR [A60T mutation], respectively). Briefly, ˜10 g samples were homogenized in ˜300 ml of cold saline using a Virtis-Tempest (Gardiner, N.Y.) apparatus. The homogenates were centrifuged at 4° C. for 30 min at 17000 rpm (37000×g) and residual saline-soluble material was removed by repeated homogenization and washing until the resultant supernatant had an OD of <0.10 at A₂₈₀. The pellet then was repeatedly homogenized, washed with cold deionized water, centrifuged, and the amyloid-laden supernatants lyophilized. The chemical nature of the fibrils was determined by automated amino acid sequencing and tandem (MS/MS) mass spectrometry, as previously described (Murphy et al., 2001).

Fibril preparation: LC fibrils were generated from rV_{λ}6 molecules as follows: 15-ml plastic tubes containing 2-ml samples of freshly prepared soluble protein in PBSA (1 mg/ml) were stoppered and sealed with parafilm, placed in a Queue orbital shaker (Pierce), and agitated at 225 rpm at 37° C. Typically, maximum fibril formation occurred by ˜48 hrs, as evidenced by ThT fluorescence intensity (LeVine et al., 1995). The fibrils were harvested by centrifugation, washed ×2 with PBSA, and stored for up to 1 wk at 4° C. or maintained frozen at −20° C. TTR fibrils were formed within 1 wk under similar conditions from solutions of soluble protein in a 0.05 M sodium acetate/0.1 M KCl buffer, pH 4.4, plus 0.001 M EDTA (Lai et al., 1996). IAPP and Aβ fibrils were generated as described previously (O'Nuallain et al., 2002; 2004). Briefly, the soluble, disaggregated peptides were dissolved in PBSA (0.25 mg/ml) and incubated at 37° C. with a seed consisting of 0.1% (by weight) sonicated IAPP or Aβ fibrils (O'Nuallain et al., 2002). Based on ThT fluorescence intensity, maximum fibril formation occurred within 5 to 7 days. All fibril samples were sonicated (2×30 sec bursts) with a probe sonic disrupter (Teledyne/Tekmar, Mason, Ohio), aliquoted, and stored at −20° C.

Europium-linked immunosorbant assay (EuLISA): A dissociation-enhanced lanthanide fluoroimmunoassay utilizing europium (Eu³⁺)-streptavidin and time-resolved fluorometry (DELFIA® system, Perkin Elmer Life Sciences, Boston, Mass.) was used to detect and characterize amyloid-reactive antibodies in human sera or IGIV preparations (Diamandis et al., 1988). Unless otherwise indicated, all steps were performed at 37° C. with 100 μl per well of reagents in assay buffer (1% BSA in PBSA containing 0.05% Tween 20). Briefly, 400-500 ng of protein, peptide, or amyloid extract in 50 μl PBS was added to each of 96 wells of a high-binding, microtiter plate (EIA/RIA Easy Wash,™ COSTAR, Corning, N.Y.) and dried overnight. Next, the wells were washed ×2 with PBSA plus 0.05% Tween 20 (wash buffer) and then blocked for 1 hr with 200 μl of 1% BSA in PBSA. The plate was gently tapped dry onto paper towels, after which the wells were filled with serial 100-μl dilutions of sera or IGIV preparations and incubated for 1 hr (in studies involving protein or peptide competitors, the concentration of IGIV remained constant [˜80 nM] and these components were serially diluted [1.2-0.0002 mg/ml]). After 2 washes, a 1:5000 dilution of biotin-labeled goat anti-human IgG antibody (γ-chain specific, Sigma) was added and the plate incubated for 40 min. After washing, wells were filled with a 1:1000 dilution of Eu³⁺-streptavidin conjugate (Perkin Elmer). Forty min later, the plate was washed ×3 and a pre-warmed (22° C.), low-pH Eu³⁺ releasing enhancement solution (Perkin Elmer) was added. Eu³⁺ time-resolved fluorescence was measured 5 to 10 min later using a Perkin Elmer Victor² 1420 Multilabel Counter (excitation, 340 nm/band width, 70 nm; emission, 615 nm/band width, 8.5 nm) with a 400-μsec delay after pulsing. The amount (fM) of lanthanide released was calculated from a standard curve using known concentrations of Eu³⁺ (Europium Standard Solution, Perkin Elmer). All measurements in this and other assays were done in triplicate (error bars in the figures represent SD).

### Results

Using an ELISA-based method, it was demonstrated previously that AL amyloidoma-bearing mice developed IgG antibodies that recognized homologous, as well as other LC-related fibrils. Further, using this technique, it was found that sera from some normal humans also contained such molecules. In an effort to determine if these antibodies recognized fibrils formed from amyloidogenic precursor proteins other than LCs, another detection system that would provide increased sensitivity and dynamic range; namely, time resolved fluorometry was utilized (Diamandis et al., 1988). This technology involves the use of a chelated lanthanide label (i.e., Eu³⁺) with unique properties, including a long excited state lifetime such that the fluorescence emission occurs several orders of magnitude after that of non-specific background (>100 μsec vs. ˜10 nsec), a large Stoke's shift (excitation, 340 nm/band width 70 nm; emission, 615 nm/band width, 8.5 nm), and a sharp emission peak of high fluorescence intensity. As detailed in the Materials and Methods section, this Eu³⁺-based, sandwich-type fluoroimmunoassay (EuLISA) involves first, the capture of reactive antibodies onto fibril-coated wells and then, their detection by sequential additions of a biotinylated anti-human IgG reagent, a streptavidin Eu³⁺ complex, a Eu³⁺-releasing agent, and, finally, measurement of fibril-bound antibody based on the magnitude of Eu³⁺ time-resolved fluorescence.

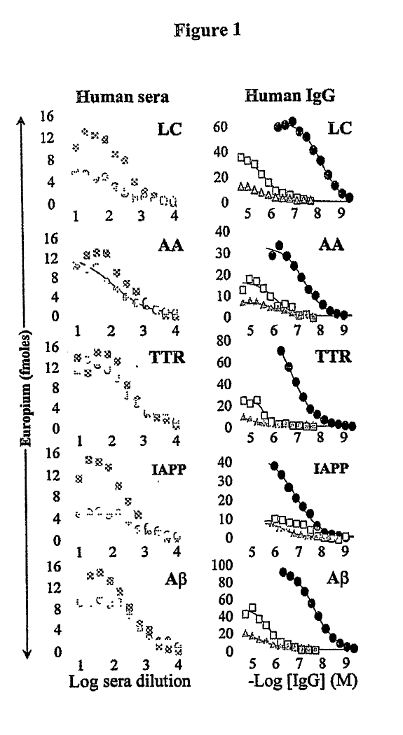

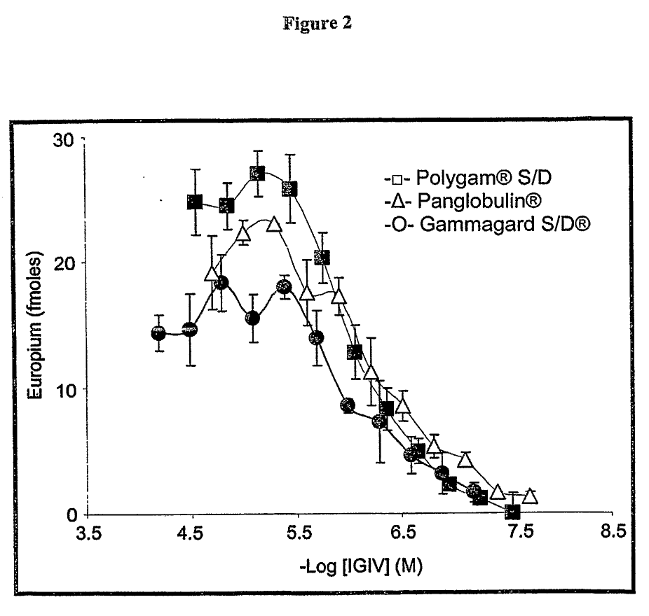

This EuLISA was used to ascertain if human sera contained IgG antibodies that recognized LC, as well as other types of fibrils, including those formed from SAA, TTR, IAPP, or Aβ. Analyses of specimens obtained from 6 healthy adults revealed that reactive molecules were detected in all; however, differences in antibody titer and amplitude of binding were apparent (FIG. 1). Based on these results, Cohn Fraction II γ-globulin and 3 different pharmacologic sources of IGIV, Gammagard S/D,® Polygam® S/D, and Panglobulin®, were assayed for the presence of LC fibril-binding antibodies. As shown in FIG. 2, these were present in all 3 products (in addition to Cohn Fraction II); further, they reacted with the 4 other types of fibrils (not illustrated).

### Example 2

### Isolation and Characterization of Fibril-Reactive IgG Antibodies Contained in Human Immune Globulin Preparations

### Material and Methods

Preparation of fibril Affinity Column: Fibrils Generated from Soluble Synthetic LCs (rV_{λ}6 Jto) were linked covalently to an N-hydroxysuccinimide (NHS)-activated Sepharose®4 fast-flow pre-activated agarose matrix with a mean bead size of 90 μm (Amersham Biosciences Corp., Piscataway, N.J.). A 10-ml packed bed volume of matrix (supplied as a suspension in 100% isopropanol) was washed three times with an equal amount of cold 1 mM HCl and centrifuged at 1000×g for 4 min at 4° C. Ten-ml of sonicated fibrils in PBS (3 mg/ml) were added to the medium and the mixture stirred gently at room temperature every 30 min. The coupling reaction was terminated 3 hrs later by addition of 0.1 M Tris-HCl, pH 7.5, to the centrifuged medium and, after another 3 hrs, the matrix was washed ×5, with each cycle consisting of 3 column volumes of 0.1 M Tris-HCl, pH 8.2, and one of 0.1M sodium acetate, pH 3.5. The final product was poured into a plastic polypropylene column (Pierce), washed four times with 10 ml of PBS, and stored at room temperature.

Preparation Of fibril affinity-Purified (Enriched) Human Immune Globulin intravenous (IGIV). As per manufacturer's instructions, lyophilized IGIV (5 g) was reconstituted with 100 ml of diluent (sterile water for injection [USP]). The sample was filtered to render the preparation aggregate-free, diluted 5-fold with PBS to yield a final concentration of 10 mg/ml, and loaded onto the fibril conjugated, PBS pre-equilibrated column. To remove any weakly binding or unbound (residual) protein, the column was washed with 40 ml of PBS and then the fibril-bound antibodies eluted in 1-ml portions using 0.1 M glycine buffer, pH 2.7; the fractions were neutralized by addition of 1 M Tris-HCl, pH 9. The concentration of IgG in the fibril affinity-purified eluates and residual filtrates was determined based on absorbance at 280 nm, using an E₂₈₀^{1%} of 1.30 and a mol wt of 150000 daltons. Samples containing the enriched antibodies were pooled and concentrated with a PL-30 Centricon® (Millipore) apparatus and stored at 4° C. for up to 2 wks or maintained frozen at −20° C.

Fibrillogenesis assay: ThT fluorescence intensity was measured to determine if IGIV preparations could block the conversion of soluble protein or peptides into amyloid fibrils. For studies involving LC (rV_{λ}6 Wil) or IAPP, wells of an ultra low-binding plate (COSTAR) were filled first with 50 μl of serially diluted (1 μM-500 nM) enriched or unfractionated IGIV in PBSA and then with 50 μl (10-100 μM) of the native amyloidogenic precursor; ThT was added to all wells to a final concentration of 30 μM and the plates sealed and incubated at 37° C. Control wells contained IGIV preparations or precursor protein/peptide (plates with LCs were shaken at 700 rpm using the THERMO star shaker [BMG LABTECH, Offenburg, Germany]). In the case of Aβ, 90 μM of soluble peptide and 1 μM of enriched or unfractionated IGIV were added to Eppendorf tubes. The mixture was incubated at 37° C. and at designated time intervals, aliquots were removed and placed in microtiter plate wells, followed by addition of ThT. Fluorescence intensity was measured daily (excitation, 440 nm/band width, 30 nm; emission, 485 nm/band width, 20 nm) with a FL600 microplate reader (Bio-Tek Instruments, Inc., Winooski, Vt.). Fibril growth was monitored to equilibrium, i.e., until maximum fluorescence was obtained.

Fibril extension assays: Biotinylated amyloidogenic molecules (rV_{λ}6 Wil and the Aβ₁₋₄₀ Cys-1 analog (O'Nuallain et al., 2004) [furnished by Dr. Ron Wetzel]) were used to determine the inhibitory effects of IGIV on fibril extension (for IAPP, the reaction was monitored by ThT fluorescence (Naiki et al., 1999)). rV_{λ}6 Wil, at a concentration of 1.0 mg/ml was labeled with a 12-fold molar excess of a 10 mM stock solution of the EZ-Link® Sulfo-NHS-Biotin® (SHB) reagent (Pierce) and incubated at room temperature for 45 min. Unreacted SHB was removed by dialysis overnight at 4° C. using a Slide-a-Lyser (Pierce) cassette with a mol wt cutoff of 3500 daltons and the concentration of the biotinylated product determined with the Micro BCA® Protein Assay Kit (Pierce). Wells of activated high-binding microtiter plates (COSTAR) each were filled with 50 μl of an 8 μg/ml suspension of sonicated LC, AD, or IAPP fibrils in PBSA and the solutions left uncovered and dried by incubation at 37° C. for 18 hrs. The wells were washed ×2 with PBSA containing 0.05% Tween 20 (wash buffer), blocked by addition of 1% gelatin in PBSA (200 μl/well) for 1 hr at 37° C., and then filled with 50-μl aliquots of serially diluted IGIV protein (4 μM-31 nM) in wash buffer. Immediately afterwards, 50 μl of soluble biotinylated LC protein (250 nM) or Aβ peptide (50 nM), as well as unbiotinylated IAPP (28 μM), in wash buffer was added and the plate incubated for 3 hrs. For experiments with LC and Aβ, the wells were filled after washing ×2, with 100 μl of a 1:1000 dilution of the Eu³⁺ streptavidin conjugate in blocking buffer containing 0.05% Tween-20, followed by another 45-min period of incubation. After 3 washes, Eu³⁺, that had been released from streptavidin by addition of enhancement solution (100 μl), was measured by time-resolved fluorometry and quantitated using a Eu³⁺ standard curve. In the case of IAPP, 50 μl of ThT (60 μM) was added to unwashed wells and ThT fluorescence measured.

Immunohistochemistry: Areas of amyloid deposition in autopsy-derived tissues were identified by Congo red staining and microscopic evaluation (Westermark et al., 2002). Immunohistochemical analyses were performed with the Elite® ABC kit, as specified by the manufacturer (Vector laboratories, Inc., Burlingame, Calif.). Deparaffinized serial 4-μm-thick sections, mounted on poly-L-lysine-coated slides, were subjected first to antigen retrieval (incubation for 30 min in boiling distilled water) and then endogenous peroxidase activity blocked by a 30-min exposure to 0.3% H₂O₂. After washing with PBS, an ˜0.3 mg/ml solution of antibody (fibril affinity-purified [enriched] IGIV or a control monoclonal IgG myeloma protein) that had been pre-mixed for 30 min at 4° C. with an appropriate dilution of goat anti-human IgG biotinylated conjugate (Vector) was added. After 48 hrs at 4° C., the sections were washed with PBS and exposed for 30 min to the ABC reagent (Avidin DH horseradish peroxidase H complex) and, after washing, to the peroxidase substrate solution, followed by a hematoxylin counterstain.

### Results

To isolate the active species from a pharmacologic source of IGIV (Gammagard S/D®), an affinity column in which Sepharose beads were conjugated with LC (rV_{λ}6 Jto) fibrils was used. Passage of the reconstituted, filtered immune globulin solution (10 mg/ml) through a 10-ml volume column yielded 2 fractions: the first consisted of unbound protein present in the PBS filtrate and the second, the fibril-bound antibody eluted by the acidic buffer (designated “residual” and “enriched” IGIV, respectively). The distribution of the 4 IgG subclasses (IgG1, 2, 3, and 4) in both pools was comparable and similar to that found in normal serum (data not illustrated). Based on the protein concentrations of the filtrate and eluate, the recovered fibril-reactive antibodies represented ˜0.2% of the immune globulin passed through the column, i.e., ˜10 mg from a bottle containing 5 g of IGIV. For further study, the enriched protein fraction was reduced in volume by ultrafiltration to a final concentration of 1 mg/ml.

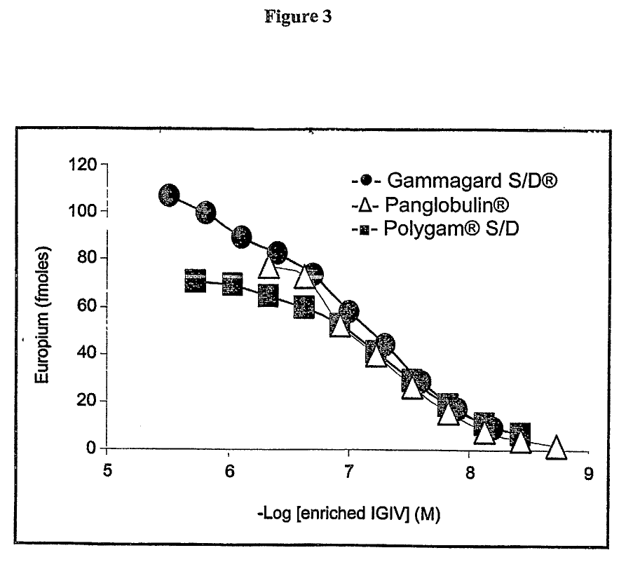

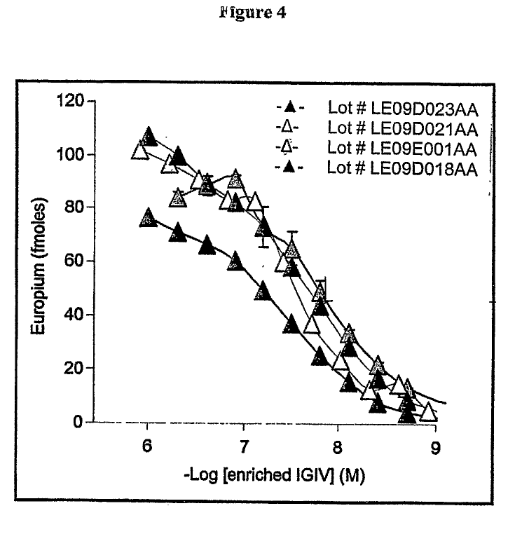

The binding of the affinity-purified antibodies to LC, AA, TTR, IAPP, and Aβ fibrils was considerably greater than that of the unfractionated human immune globulin preparation, thus indicating that virtually all of the fibril-reactive material was isolated by the chromatographic procedure (FIG. 1). The EC50 value (antibody concentration that gives half maximum binding) of the affinity purified, enriched IGIV preparation for LC and Aβ fibrils (˜15 nM) was ˜70 and 200 times less than that of the unfractionated (˜1 μM) and antibody depleted (˜3 μM) materials, respectively (these values remained unchanged when enriched or residual fractions were retested after storage at 4° C. for 1 week). For AA, the EC50 of the enriched fraction was ˜23 times less (46 nM) than that of the unfractionated (1 μM). In the case of TTR and IAPP, the EC50 was not obtained; however, comparable binding was noted. Anti-fibril antibodies with equivalent EC50 values to Gammagard S/D® were present in the other 2 pharmacologic sources of human immune globulin, Polygam® S/D, and Panglobulin® (FIG. 3) and no appreciable differences in titer were found when the enriched IgG fraction from multiple lots of Polygam® S/D was tested (FIG. 4).

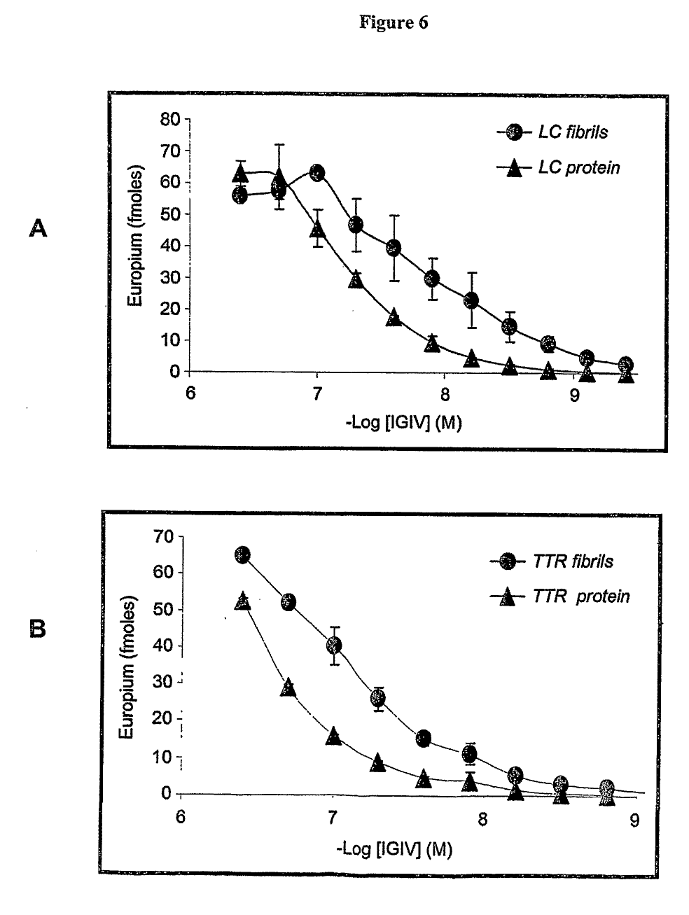

As shown in FIG. 5, the affinity-purified IGIV reacted specifically with LC, TTR, and Aβ fibrils, but remarkably, did not recognize their non-fibrillar, soluble counterparts (even in an up to 125 molar excess). Further, in a competition EuLISA, LC, TTR, and IAPP fibrils were effective inhibitors of this interaction (FIG. 15C). In addition to the specificity of the enriched IGIV for fibrils, these molecules had equivalent affinity for partially denatured amyloidogenic precursors (FIG. 6) that were generated when LCs or TTR were dried onto wells (Friguet et al., 1984; Höök et al., 1998) (the non-fibrillar nature of this material was evidenced by the fact that they were ThT negative). In studies with human serum or other soluble proteins (thyroglobulin, collagen, insulin), as well as non-amyloidogenic aggregated components (ds DNA, reduced and alkylated ovalbumin (O'Nuallain et al., 2002), elastin), the antibodies did not react with these components when they were tested at concentrations of up to 0.5 mg/ml and 1.5 mg/ml, respectively (not illustrated).

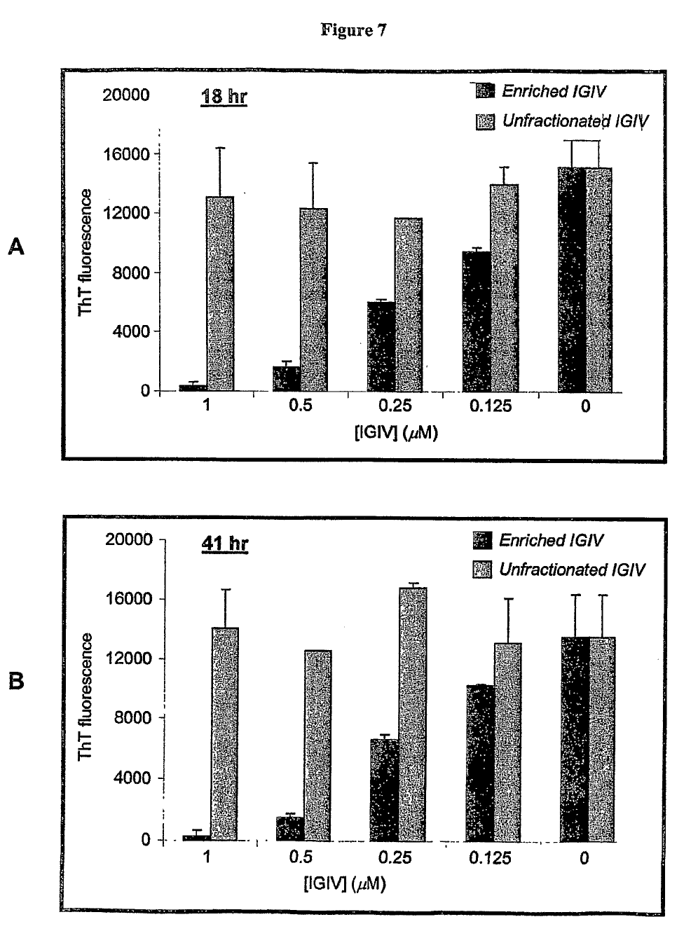

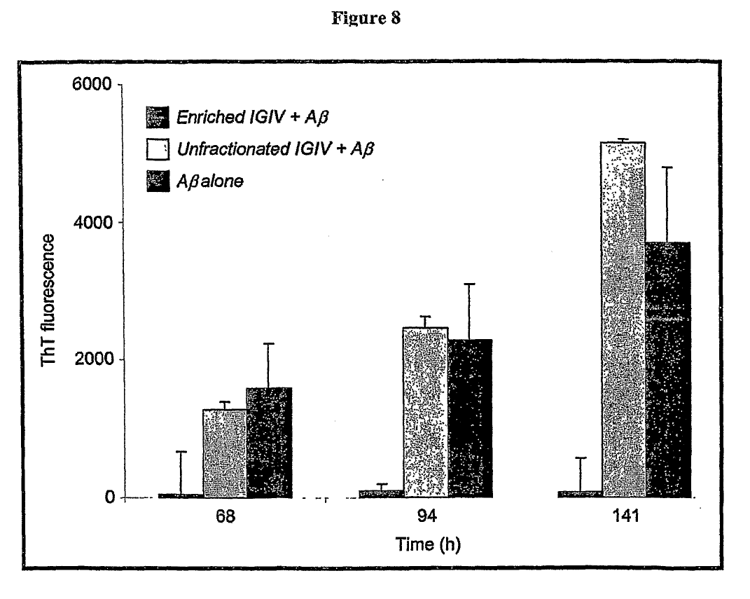

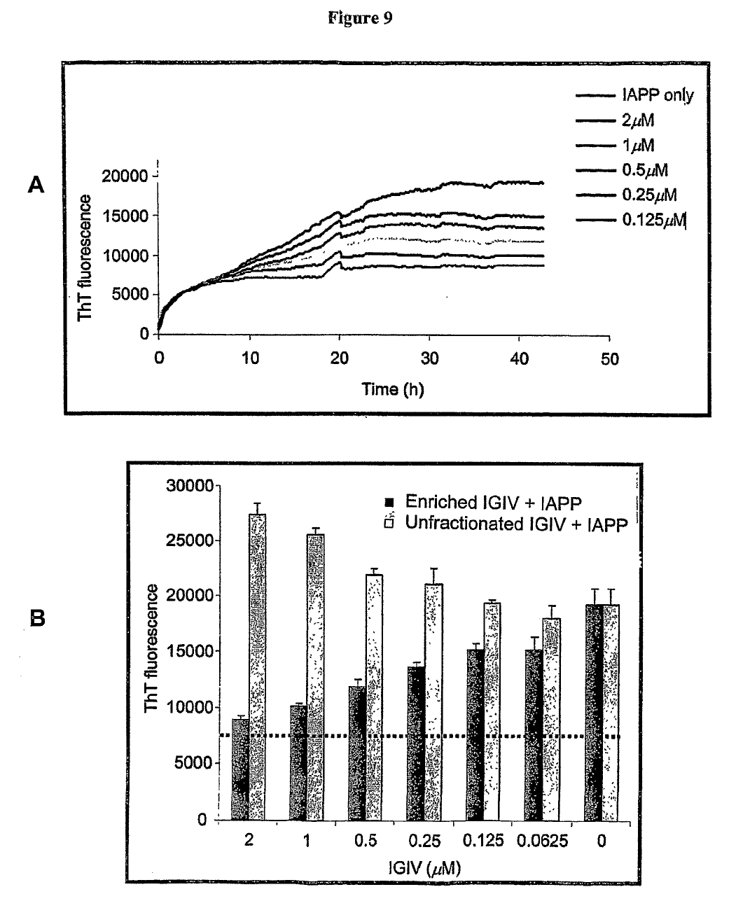

In other experiments, it was demonstrated that IGIV anti-fibril antibodies could inhibit fibrillogenesis, as evidenced in fibril formation and fibril extension assays. In the case of the former, co-incubation of sub-equimolar amounts of the enriched fraction with the soluble amyloidogenic LC-related (rV_{λ}6 Wil) protein markedly decreased the generation of ThT-positive material, i.e., fibrils, with an IC50 value (antibody concentration that gives 50% inhibition) of 250 nM, while the unfractionated IGIV had no effect (FIG. 7). This was not a transient, kinetic response, as inhibition was evidenced at the end of the reaction (˜18 hr) and persisted over at least an ˜4-day period. Similarly, these molecules blocked Aβ fibril formation (FIG. 8). As for IAPP, based on kinetic traces, the enriched IGIV antibodies slowed fibrillogenesis by halting conversion of the less structured intermediate form (O'Nuallain et al., 2004; Higham et al., 2000) into fibrils (IC50, 0.25 μM), as evidenced by the single and biphasic natures of the curves generated in the presence or absence of IGIV, respectively (FIG. 9). In these studies, changes in ThT fluorescence emission indicative of the inhibition of de novo fibrillogenesis could not be attributed merely to non-specific suppression, in as much as the same signals were obtained when the antibodies were incubated with pre-formed fibrils.

The inhibitory capability of affinity-purified IGIV also was evidenced by electron microscopy. Samples of native LC (V_{λ}6 Wil, 5 μM), IAPP (50 μM), and Aβ (92 μM) were co-incubated with these molecules (1 μM) at 37° C. for ˜14 days, dried onto grids, and stained with uranyl acetate. In the case of LC and Aβ, the fibril-reactive antibodies totally blocked fibril formation (not illustrated). For IAPP, electron micrographs revealed less structured, densely aggregated material characteristic of the intermediate (O'Nuallain et al., 2004), whereas, in the presence of unfractionated IGIV or PBS, these preparations contained ultrastructurally typical fibrils (FIG. 16E). Further, the enriched IGIV fraction limited growth of LC, IAPP, and Aβ fibrils (FIG. 16F-16H) with IC50 values in the extension assays of 1.6 μM, 0.9 μM, and 50 nM, respectively.

The fibril affinity-purified IGIV antibodies also recognized amyloid in tissue, as documented immunohistochemically. In contrast to the control IgG myeloma protein, these molecules immunostained the green birefringent congophilic deposits present in the renal glomeruli, pericardium, myocardium, and pancreas from patients with ALκ, ALλ, ATTR, and AIAPP amyloidosis, respectively (FIG. 17). Similar reactivity was found for AA-containing tissue (not illustrated). The specificity of this interaction was further evidenced by the fact that the enriched antibodies, when absorbed overnight at 4° C. with sonicated LC fibrils, were totally unreactive.

### Example 3

### Diagnostic Potential of Fibril-Reactive IGIV Antibodies

### Materials and Methods

Radioimaging studies: Fibril affinity-purified (enriched) IGIV was labeled with I-125 (1 mCi [37 MBq] per mg of protein) using a modification of the chloramine T method (Kennel et al., 1983); residual isotope and protein aggregates were removed by size-exclusion liquid chromatography using Sephacyl AcA34 (Pharmacia). Mice bearing 50-mg human amyloid tumors received, 7 days later, a 100-μl injection into the lateral tail vein of labeled antibody in PBS containing BSA as carrier protein (5 mg/ml). To block radioiodine uptake by the thyroid gland, 1% (v/v) Lugol's solution was added to the animals' drinking water 3 days prior to antibody administration. The mice were euthanized by CO₂ inhalation (thus reducing blood pooling that can occur within the thoracic structures after cervical dislocation) 72 hrs after receiving the labeled antibody and samples of skin, muscle, abdominal fat, liver, pancreas, kidney, spleen, heart, and lung, as well as the amyloidoma, were harvested and placed into tared vials. To determine the biodistribution of the labeled reagent, specimens were weighed and the radioactivity measured in a gamma counter. The primary index values were expressed as % injected dose per gram of tissue.

Single photon emission computerized tomography (SPECT) data on amyloidoma-bearing mice were collected using a micro-SPECT instrument that incorporated a 10 mm-long hexagonal parallel-hole collimator and two detector heads, each containing a 50 mm-diameter Hamamatsu R2486-02 multi-anode photo-multiplier tube coupled to an array of 1×1×8 mm CsI (T1) crystals arranged on a 1.2 mm² grid; this imaging system was capable of high-level spatial resolution to ˜1.7 mm (Paulus et al., 2000). The SPECT detectors were placed ˜5 cm from the 50-ml conical tubes housing the mice. For each study, 60 data sets were obtained at 6° intervals and the images reconstructed using an implementation of the EM-ML algorithm (Walrand et al., 1996).

To localize anatomic regions of isotope uptake, after completion of the SPECT study, animals were placed in the high-resolution (to 50 μm) micro-CT apparatus and 180 projections were collected. To facilitate co-registering of the reconstructed SPECT and CT images, 3 capillaries filled with an I-125 solution placed on the conical tubes were used for reference purposes and provided fiducials in the x, y, and z planes.

### Results

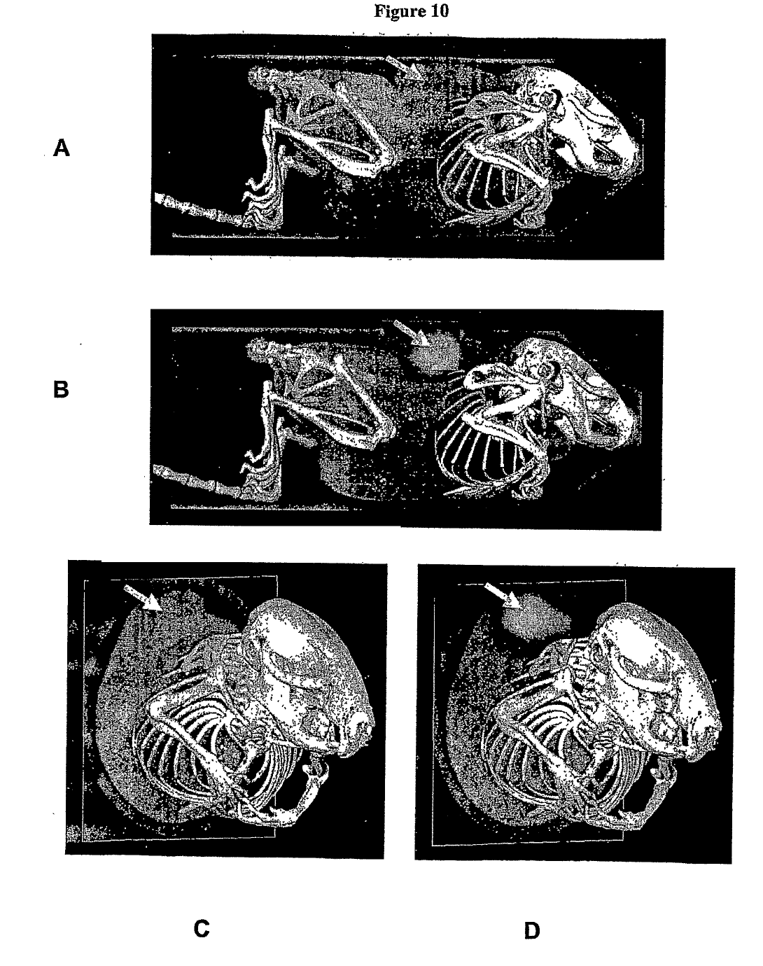

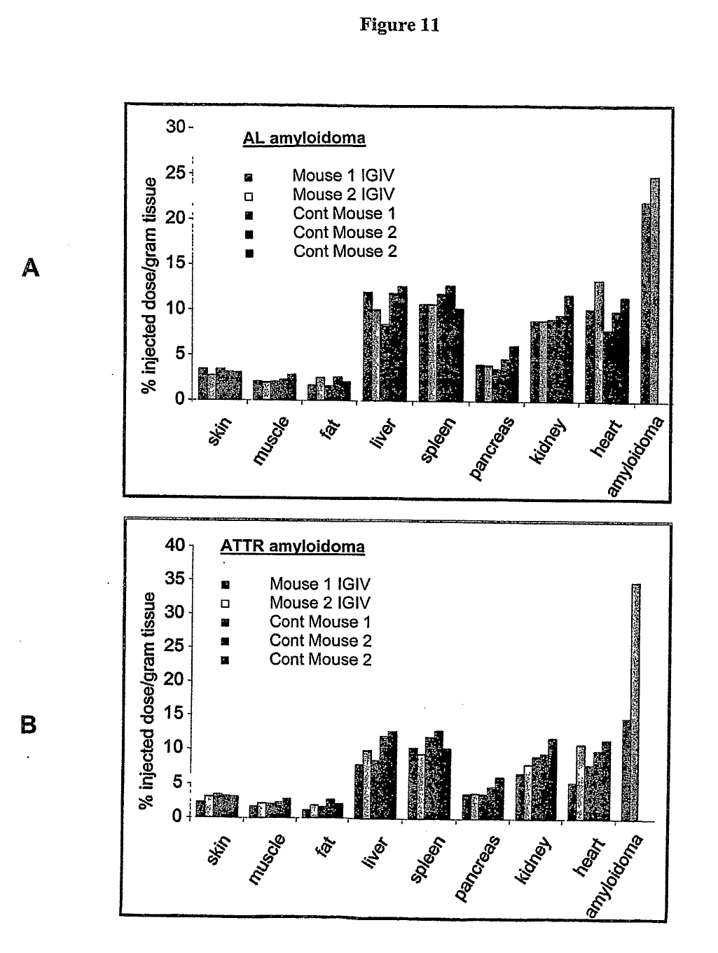

Based on the studies that showed that radioiodinated derivatives of the amyloid-reactive mAb 11-1F4 could be used to image amyloid in the in vivo murine amyloidoma model by high resolution SPECT imaging (Wall et al., 2004), this technology was used to determine whether fibril affinity-purified IGIV had a similar capability. Mice were given 15 μg (˜160 μCi, ˜6 MBq) injections into the tail vein of ¹²⁵I-labeled enriched IGIV 1 week after induction of amyloidomas containing 50 mg of human AL or ATTR extracts and euthanized 72 hrs later (non-amyloidoma bearing animals served as controls). Localization of the radioiodinated antibodies to the amyloid was apparent by micro-SPECT/micro-CT where co-registered tomographic images revealed the radioisotope to be concentrated within the AL (FIG. 10) and ATTR amyloidomas (not illustrated), with only minimal background activity occurring in other organs or tissues. The preferential uptake of the radioiodinated IGIV antibodies by the amyloid also was indicated by the biodistribution of the labeled reagent where the specific activities of the harvested tumors ranged from 12 to 35% of the injected dose per gram of tissue, a value greater than that found at any other site (FIG. 11). The uptake of radioiodinated IGIV from other areas was comparable to that of the control mice.

### Example 4

### Therapeutic Potential of Fibril-Reactive IGIV Antibodies

### Materials and Methods

Amyloidoma formation in mice: Lyophilized water-soluble AL or ATTR amyloid extracts were suspended in 25 ml of sterile saline and homogenized with a PCU-2 Polytron apparatus (Brinkman, Luzerne, Switzerland). The fibrils were sedimented by centrifugation at 6° C. for 30 min at 10000 rpm (12000×g) and 50 to 100 mg of the resultant pellet was re-suspended in 1 ml of sterile saline and rehomogenized. This solution was injected s.c. between the scapulae of 8 wk-old SCID or BALB/c mice (Taconic, Germantown, N.Y. and Charles Rivers, Wilmington, Mass., respectively) using an 18-gauge needle attached to a 6-mil syringe. The size of the resultant amyloidoma was measured by daily palpation and the weight established at necropsy. The mice were treated in accordance with National Institutes of Health regulations under the aegis of a protocol approved by the University of Tennessee's Animal Care and Use Committee.

### Results

Given that human AL and ATTR amyloidomas were rapidly resolved when mice were treated with the amyloid-reactive mAb-11-1F4 (Hrncic et al., 2000), it is interesting to test whether human immune globulin fibril-reactive antibodies would have a similar effect. Initially, SCID mice were injected s.c. with 100 mg of ALκ, ALλ, or ATTR extracts and then given in the tail vein, on days 0, 2, 4, 6, and 8, a series of 100-μl injections containing 100 μg of either fibril affinity-purified IGIV, a human monoclonal (myeloma) IgG protein, or the mAb 11-1F4; for comparison, amyloid-bearing mice received an equivalent volume of PBS. Only the mAb was effective in promoting resolution of the amyloidoma. Subsequently, the dose of the enriched IGIV fraction was increased 5-fold (0.5 mg in 0.5 ml) and given s.c. in 2 sites every 48 hrs×5 (for control purposes, amyloid-bearing mice received an equivalent volume of PBS). As shown in FIG. 18, there was marked reduction in the size of the induced ALκ, ALλ, and ATTR amyloidomas (90, 99, and 65%, respectively) in the enriched antibody-treated mice, as compared to animals given PBS or unfractionated IGIV alone.

### Example 5

### Clinical Applicability of Fibril-Reactive IGIV Antibodies

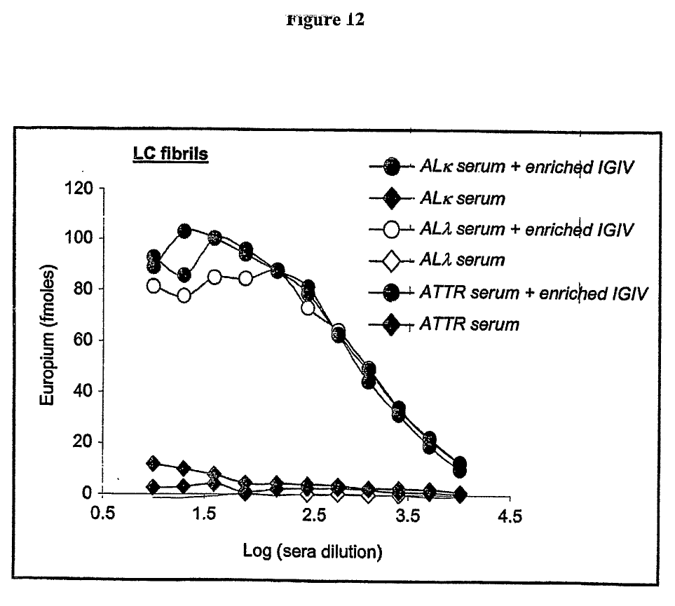

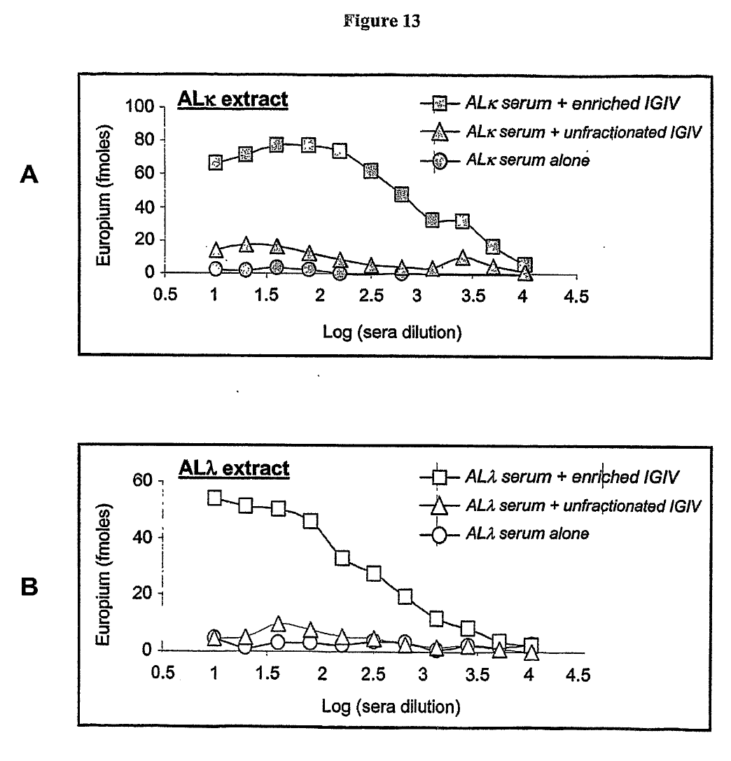

Analyses of sera from patients with amyloidosis, particularly AL, revealed them to contain low titers of fibril-reactive antibodies; however, this activity could be increased significantly when enriched IGIV was added to the specimens (FIG. 12). In the case of AL, this enhanced reactivity was evidenced not only against synthetic LC fibrils, but also to protein extracted from the patients' amyloid-laden tissue (FIG. 13). Further, these antibodies recognized and reacted with comparable affinity to ALκ and ALλ extracts, regardless of the V_{L} gene family (data not illustrated).

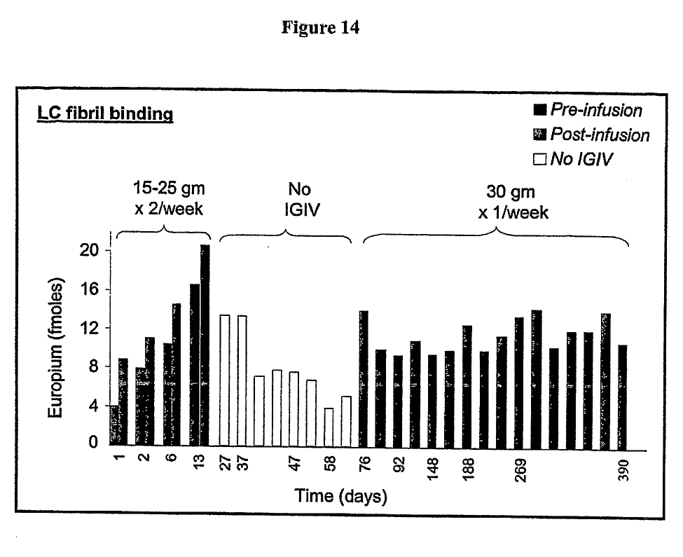

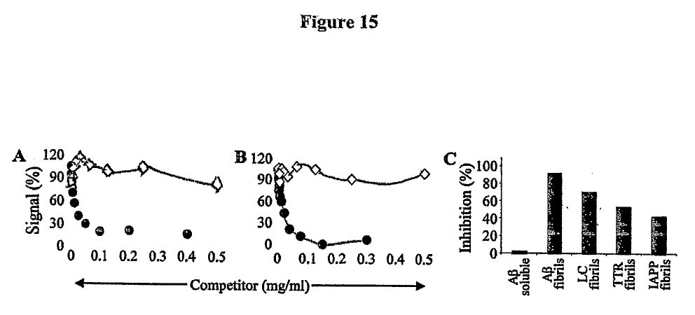

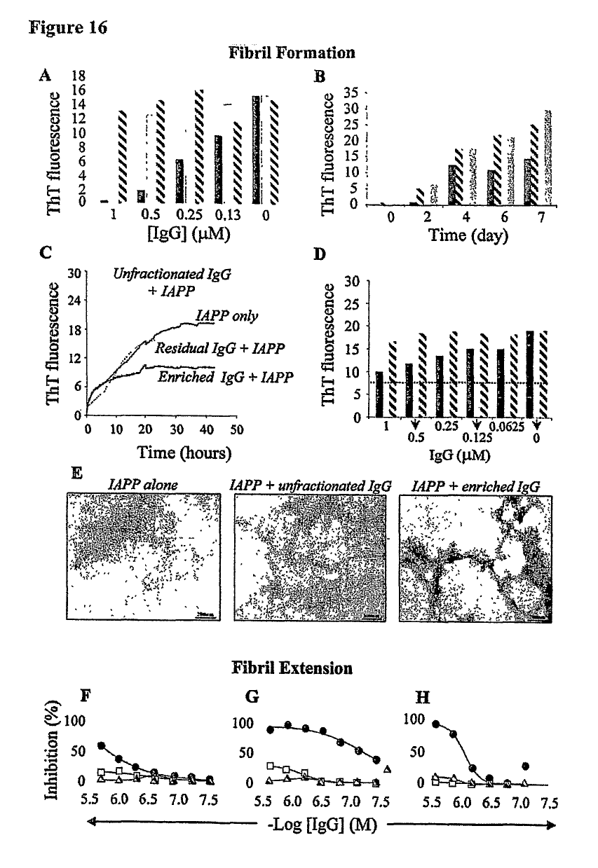

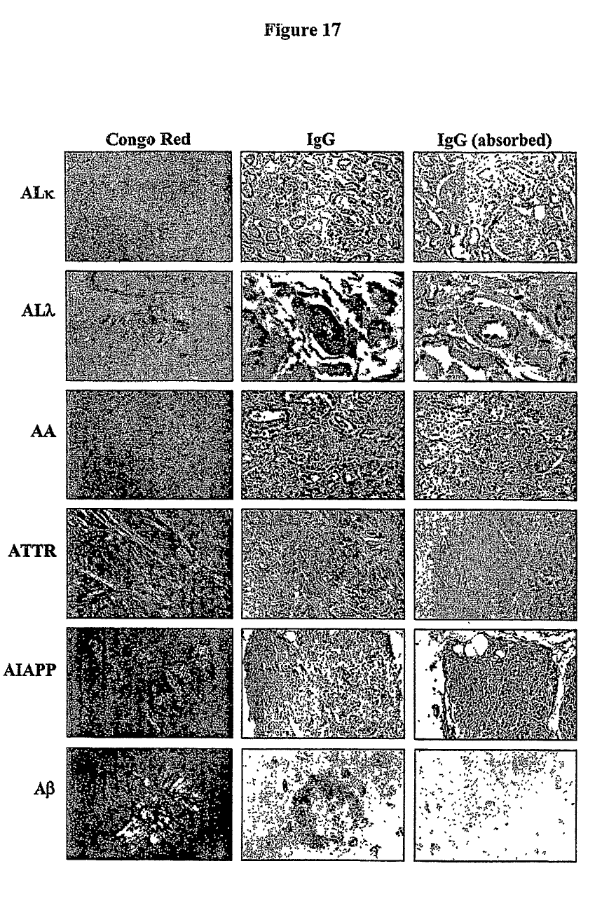

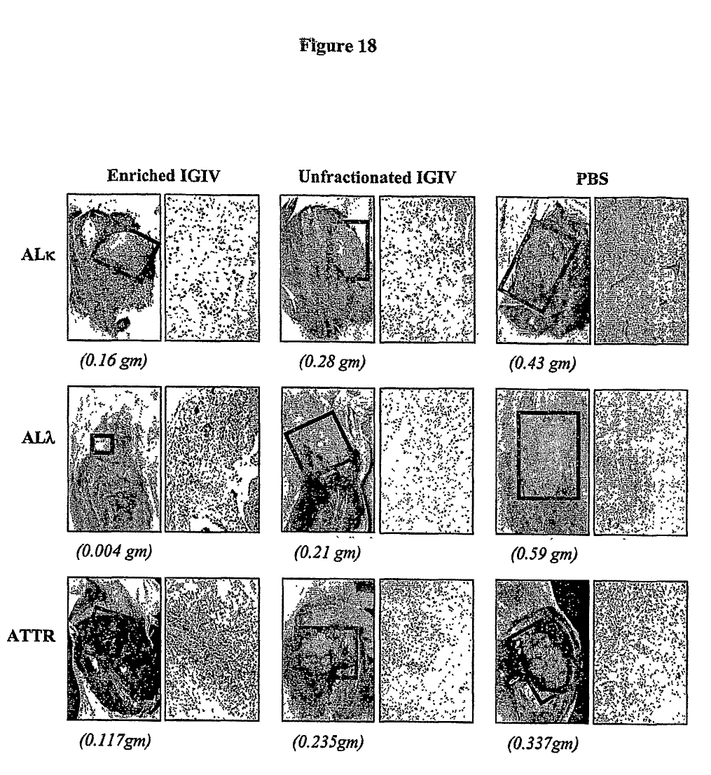

To determine if human immune globulin infusions could have a similar effect in vivo and to obtain pharmacokinetic data, an ALλ patient was given IGIV infusions (Gammagard S/D®) over a period of 12 months. The treatment was well-tolerated and resulted in an up to a 5-fold increase in serum antibody titer to synthetic LC fibrils (FIG. 14), as well as to the homologous amyloid extract (not illustrated). During a 7-week interval when no IGIV was given, an ˜50% decrease in antibody titer was evident after 14 days; however, resumption of the weekly infusions resulted in a sustained 2- to 3-fold increase in the reactivity to LC fibrils over the ensuing 6+ months.

### Example 6

### Diagnostic and Therapeutic Potential of Amyloid-Reactive IgG Antibodies Contained in Human Sera

It has been discovered for the first time that human sera, as well as immune globulin products derived from large pools of plasma from normal donors, contain IgG antibodies that also recognize a conformational epitope(s) expressed on light chains (LC), serum amyloid A (SAA), transthyretin (TTR), islet amyloid polypeptide (IAPP), and amyloid β1-40 peptide (Aβ) fibrils, but do not when these components are in their native non-fibrillar states. Affinity chromatography was employed to isolate the reactive species and the results of these studies demonstrate the capability of these antibodies to inhibit fibrillogenesis, as well as immunostain amyloid deposits. Moreover, evidence is provided that these molecules have in vivo activity in that they can be used to image amyloid and accelerate its destruction when given to mice bearing human amyloidomas. The present findings suggest that fibril-reactive IgG antibodies, due to their unique specificity and human origin, could have novel diagnostic and therapeutic potential for patients with amyloid-associated disease.

### Materials and Methods

Proteins and peptides: Human immune globulin preparations (Gammagard S/D®, Polygam®S/D, and Panglobulin®) were products of Baxter Healthcare Corp. (Westlake Village, Calif.). The murine IgGκ protein MOPC 31-C was purchased from Sigma (St. Louis, Mo.). Recombinant λ6 LC variable region (rV_{λ}6) components Jto and Wil were produced in an E. coli expression system and recombinant mutant (V30M) TTR was provided by Dr. Joel N. Buxbaum (Wall et al., 1999). The Aβ peptide, its Cys-1 analog, and IAPP were synthesized at the Keck Biotechnology Center (Yale University, New Haven, Conn.).

Amyloid fibrils used for amyloidoma formation in mice were extracted from tissue samples obtained post-mortem from patients with ALκ-, ALλ-, AA-, or ATTR (T60A mutation)-associated amyloidosis and their chemical nature determined by automated amino acid sequencing and tandem (MS/MS) mass spectrometry (Murphy et al., 2001).

Soluble rV_{λ}6 molecules were biotinylated with a 12-fold molar excess of a 10 mM stock solution of EZ-Link® Sulfo-NHS-Biotin® (Pierce) and, after incubation at room temperature for 45 minutes, the unreacted SHB was removed by dialysis. The biotinylated AD Cys-1 analog was provided by Dr. Ron Wetzel.

LCs were sterile-filtered using a 0.22 μm PVDF 25 mm Millex®-GV syringe-driven filter unit (Millipore, Beillerica, Mass.) and found by Sephadex G25 (Amersham Biosciences, Corp., Piscataway, N.J.) gel filtration to consist of monomers and dimers; no higher-order aggregates were present (Wall et al., 1999). The IAPP and Aβ preparations were disaggregated by sequential exposure to trifluoroacetic acid and hexafluoroisopropanol (O'Nuallain, et al., 2004). Prior to study, all proteins or peptides were centrifuged at 20,800×g for 25 minutes and, in the case of Aβ, 50,000×g for 18 hours.

Fibril preparation: In vitro generated LC, IAPP, and Aβ fibrils were prepared as described previously (O'Nuallain, et al., 2002). TTR fibrils were formed within 7 days under similar conditions from solutions of soluble protein (˜2 mg/ml) incubated at 37° C. in a 0.05 M sodium acetate/0.1 M KCl buffer, pH 4.4, plus 0.001 M EDTA (maximum ThT signal was obtained at this time-point). All fibril samples were harvested by centrifugation, sonicated (2×30 s bursts) with a probe sonic disrupter (Teledyne/Tekmar, Mason, Ohio), aliquoted, and stored at 4° C. for up to 2 weeks or long-term at −20° C. These synthetic fibrils were used in all experiments except the amyloidoma studies.

Preparation of fibril affinity column: Ten ml of LC (rV_{λ}6 Jto) sonicated fibrils in PBS (3 mg/ml) were conjugated at room temperature for 3 hours to a 10-ml packed bed volume of N-hydroxysuccinimide-activated Sepharose® 4 fast-flow agarose matrix (Amersham Biosciences Corp.) and the column equilibrated with PBS.

Isolation of fibril reactive IgG antibodies: Lyophilized immune globulin (5 g) was reconstituted in 100 ml of sterile water for injection (USP). The sample was passed through a 0.22 μm filter, diluted 5-fold with PBS to yield a final concentration of 10 mg/ml, and loaded onto the fibril-conjugated column. Weakly binding or unbound (residual) protein was collected and then the column was washed with PBS and the fibril-bound (enriched) antibodies eluted in 1-ml portions using 0.1 M glycine buffer, pH 2.7; the fractions were neutralized by addition of 1 M Tris HCl, pH 9. The concentration of IgG in each sample was determined based on absorbance at 280 nm, using an E^{1%} of 1.30 and a Mᵣ of 150,000 Da. Samples containing the enriched antibodies were pooled and concentrated to 1 mg/ml with a PL-30 Centricon® (Millipore) apparatus.

The IgG subclass composition of the affinity-purified antibodies was determined using the Human IgG Subclass Profile ELISA Kit (Zymed® Laboratories, San Francisco, Calif.).

Europium-linked immunosorbant assay (EuLISA): The dissociation-enhanced lanthanide fluoroimmunoassay (Diamandis, E.P., 1988) incorporating europium (Eu³⁺)-streptavidin and time-resolved fluorometry (DELFIA® system, Perkin Elmer Life Sciences, Boston, Mass.) was essentially as described in O'Nuallain, et al., 2002, except for the reduced concentration of BSA (1%) used for blocking and in the assay buffer. For the binding studies, sera or IgG fractions were serially diluted in activated, high-binding microtiter plate wells (COSTAR, Corning, N.Y.) coated with 400-500 ng of soluble or fibrillar protein or peptide, respectively. For competition studies, the concentration of antibody remained constant (˜80 nM) and inhibitors were serially diluted (1.2-0.0002 mg/ml). A biotinylated goat anti-human IgG reagent (γ-chain specific, Sigma) served as secondary antibody and, after addition of a Eu³⁺-streptavidin conjugate followed by the releasing enhancement solution, Eu³⁺ time-resolved fluorescence was measured with a Perkin Elmer Victor² 1420 Multilabel Counter. The amount (fM) of lanthanide released was calculated from a standard curve using known concentrations of Eu³⁺. All measurements in this and other assays were done in triplicate (error bars in the figures represent SD).

Fibrillogenesis assay: ThT fluorescence intensity was monitored to determine if IgG preparations could block conversion of soluble proteins or peptides into amyloid fibrils. For studies involving LC (rV_{□}6 Wil) or IAPP, wells of an ultra low-binding plate (COSTAR) were filled first with 5 or 50 μM, respectively, of the native molecules and then with serially diluted (0.5 μM-250 nM) enriched, residual, or unfractionated antibody in PBS containing 0.05% sodium azide (PBSA) plus 30 μM ThT (control wells contained IgG preparations or precursor protein/peptide). The plates were then sealed and incubated at 37° C. Fluorescence was measured daily with a FL600 microplate reader (Bio-Tek Instruments, Inc., Winooski, Vt.). In the case of Aβ, 90 μM of soluble peptide and 1 μM of enriched, residual, or unfractionated IgG were added to Eppendorf tubes and at designated time intervals, aliquots were removed and placed in microtiter plate wells, followed by addition of ThT.

Fibril extension assays: Wells of activated high-binding microtiter plates each were filled with 400 ng of sonicated LC, Aβ, or IAPP fibrils in PBSA and dried as described (O'Nuallain, et al., 2004). The wells were blocked by addition of 1% gelatin in PBSA for 1 hour at 37° C. and then filled with 50 μl of serially diluted IgG (4 μM-31 nM) in wash buffer (PBS plus 0.02% Tween 20). Immediately afterwards, 50 μl of soluble biotinylated LC protein (250 nM), Aβpeptide (50 nM), or unbiotinylated IAPP (28 μM) was added and the plate incubated for 3 hours. For experiments with LC and Aβ, the wells were filled with the Eu³⁺-streptavidin conjugate, then with enhancement solution, and free Eu³⁺ was quantitated by time-resolved fluorometry. In the case of IAPP, ThT was added to reaction wells and its fluorescence monitored.

Immunohistochemistry: Areas of amyloid deposition in autopsy-derived human tissues were identified by Congo red staining and microscopic evaluation (Westermark, et al., 1999). Immunohistochemical analyses were performed with the Elite® ABC kit (Vector laboratories, Inc., Burlingame, Calif.). Deparaffinized serial 4-μm-thick sections, mounted on poly-L-lysine-coated slides, were subjected to antigen retrieval (30 min in boiling distilled water) and then endogenous peroxidase activity blocked with 0.3% H₂O₂. After washing with PBS, the tissues were exposed to fibril-reactive antibody (0.1 mg/ml) that had been pre-mixed with an appropriate dilution of goat anti-human IgG biotinylated conjugate (for control purposes, the antiserum also was absorbed with a 10-fold excess of sonicated LC fibrils) and the slides incubated for 48 hours at room temperature. Sections were washed, subjected first to the avidin DH horseradish peroxidase H complex, and then to the peroxidase substrate solution, followed by a hematoxylin counterstain.

For studies involving cerebral cortical tissue from a patient with Alzheimer's disease, amyloid deposits in 6 μm-thick sections were identified after Congo red staining and confirmed to contain Aβ-related protein immunohistochemically (after pre-treatment for 5 minutes at room temperature with 95% formic acid) using a murine anti-Aβ mAb (NCL-B-amyloid, Novacostra Laboratories Ltd, Newcastle upon Tyne, United Kingdom) in conjunction with the Vector Immuno PRESS detection system (Vector). Antigen retrieval involved heating the specimen at 90° C. for 20 seconds in Dulbecco saline solution, pH 7.4, using a Digital Decloaking Chamber (Biocare Medical, Concord, Calif.), as per manufacturer's instructions, followed by exposure first to 4% sodium dodecyl sulfate for 1 hour at 37° C. and then to a 0.25% solution of pepsin (Pepsin Kits, Bio Genex, San Ramon, Calif.) for 30 minutes at 37° C. For immunostaining, sections were incubated for 18 hours at room temperature with the enriched IGIV preparation [0.02 mg/ml]) that had been pre-mixed with an appropriate dilution of a goat anti-human IgG biotinylated conjugate (Vector). After washing with PBS, the murine anti-Aβ mAb-treated tissue was overlaid with an HRP-conjugated secondary antibody followed by addition of the substrate-chromagen solution. The IGIV-treated section was subjected first to the Avidin DH-HRP-H complex, then to the peroxidase substrate (Elite® ABC kit, Vector), and finally, as a counterstain, to hematoxylin.

Amyloidoma formation in mice: Eight week-old BALB/c or SCID mice were injected s.c. between the scapulae with 50 to 100 mg of human AL or ATTR extracts, as previously described (Hrncic, et al., 2000). The animals were treated in accordance with NIH regulations under the aegis of a protocol approved by the University of Tennessee's Animal Care and Use Committee.

Radioimaging studies: Fibril affinity-purified (enriched) IgG antibodies were labeled with I-125 (1 mCi [37 MBq] per mg of protein) by the chloramine T method; residual isotope and protein aggregates were removed by size-exclusion liquid chromatography. Imaging data were recorded using a micro single photon emission computed tomography (SPECT) instrument capable of high-level spatial resolution to 1.7 mm (Paulus et al., 2000). For each study, 60 projections were acquired at 6° intervals and the images reconstructed using an implementation of the EM-ML algorithm (Walrand et al., 1996). After completion of the SPECT study, animals were placed in the high-resolution (to 50 μm) microCT apparatus and 180 projections were collected. Reconstructed SPECT and CT images were co-registered using 1-125 filled capillaries as fiducials.

To determine the biodistribution of the labeled reagent, samples of skin, muscle, abdominal fat, liver, pancreas, kidney, spleen, heart, and lung, as well as the amyloidoma, were harvested, placed into tared vials, and the radioactivity measured. The primary index values were expressed as % injected dose per gram of tissue.

Statistical analyses: Data were compared using a one-way analysis of variance with confidence limits of 95%.

Assurances: Studies involving human specimens were in accordance with a protocol approved by the University of Tennessee Medical Center's Institutional Review Board.

### Results

Initially an ELISA-based method was used to demonstrate that AL amyloidoma-bearing mice developed IgG antibodies that recognized homologous, as well as other LC-related fibrils (Hrncic et al., 2000). Further, with this technique, it was discovered that sera from some normal humans also contained such components. In an effort to determine if these antibodies bound to fibrils formed from amyloidogenic precursor proteins other than LCs, another detection system that would provide increased sensitivity and dynamic range was used; namely, time resolved fluorometry (Diamandis, E.P., 1988) that utilizes the chelated lanthanide label europium (Eu³⁺). As detailed in the Materials and Methods section, this sandwich-type fluoroimmunoassay (EuLISA) involves the capture of reactive antibodies onto fibril-coated wells and their detection by sequential addition of a biotinylated anti-human IgG reagent, a streptavidin Eu³⁺ complex, and a Eu³⁺-releasing agent. Finally, the amount of fibril-bound antibody is determined based on the magnitude of Eu³⁺ fluorescence.

Analyses by EuLISA of serum specimens obtained from 10 healthy adults revealed varying titers of IgG antibodies that recognized LC fibrils. Additionally, the 5 most reactive samples also bound those formed from SAA, TTR, IAPP, or Aβ. The results obtained in 2 representative cases are illustrated in FIG. 1. On this basis, Cohn Fraction II γ-globulin and three different pharmacologic sources of immune globulin (IGIV) were assayed and it was discovered that fibril-binding antibodies were present in all.

To isolate the active species from IGIV, affinity chromatography where LC fibrils were conjugated to Sepharose beads was used. Passage of the reconstituted, filtered solution through the column yielded two fractions: the first consisted of unbound protein present in the PBS filtrate and the second, the fibril-bound antibody eluted by the acidic buffer (designated “residual” and “enriched” IgG, respectively). The distribution of the IgG 1, 2, 3, and 4 subclasses in both pools was comparable and similar to that found in normal serum. Based on the protein concentrations of the filtrate and eluate, the isolated antibodies represented ˜0.2% of the IgG molecules in the IGIV product.

The affinity of the purified antibodies for LC, AA, TTR, IAPP, and Aβ fibrils was considerably greater than was that of the unfractionated preparation, thus indicating that virtually all of the fibril-reactive IgG was isolated by the chromatographic procedure (FIG. 1). The EC50 binding value of the affinity purified preparation for LC and Aβ fibrils (˜15 nM) was ˜70 and 200 times less than that of the unfractionated (˜1 μM) and residual (˜3 μM) materials, respectively. For AA, the EC50 of the enriched fraction was ˜23 times less (46 nM) than that of the unfractionated (1 μM), while for TTR and IAPP, the EC50 was not obtained; however, comparable binding was noted. In other experiments, IgG antibodies eluted from an Aβ fibril-conjugated column reacted with comparable EC50 values to Aβ and LC fibrils as did those derived from the LC affinity matrix.

As shown in FIGS. 15A and B, affinity-purified IgG reacted specifically with LC and Aβ fibrils in a competition assay, but remarkably, did not recognize their soluble counterparts (even when tested at 125 molar excess). Further, LC, TTR, and IAPP fibrillar components were efficient inhibitors of the interaction with Aβ fibrils (FIG. 15C), whereas human sera or other soluble proteins (thyroglobulin, collagen, insulin), as well as non-amyloidogenic aggregated molecules (ds DNA, reduced and alkylated ovalbumin, elastin), at concentrations of up to 1.5 mg/ml, had no effect.

As evidenced in fibril formation and extension assays, the enriched IgG fraction effectively inhibited fibrillogenesis, as compared with the unfractionated or residual IGIV pools. In this regard, co-incubation of sub-equimolar amounts of the purified fraction with soluble amyloidogenic LC protein markedly decreased the generation of ThT-positive material, i.e., fibrils, with an IC50 value of 250 nM, while the unfractionated portion had no effect (FIG. 16A). This was not a transient or kinetic response, as the inhibition noted at 18 hours (the time at which maximum fibril formation occurred in the absence of antibody) persisted over at least ˜2 days. Similarly, these molecules blocked Aβ fibril formation (FIG. 16B). As for IAPP, based on kinetic traces, the enriched IgG antibodies slowed fibrillogenesis by halting conversion of the less structured intermediate form (O'Nuallain et al., 2004) into fibrils, as seen by the single and biphasic nature of the curves generated in the presence or absence of antibody, respectively (FIG. 16C). Additionally, in contrast to residual or unfractionated IgG, the isolated preparation inhibited IAPP fibrillogenesis in a dose-dependent manner with an IC50 value of 0.25 μM (FIG. 16D). Control experiments with pre-formed LC, Aβ, and IAPP fibrils indicated that the fluorescence emission from bound ThT was not quenched non-specifically by the antibody.

The inhibitory capability of affinity-purified IgG also was shown by electron microscopy. Samples of native LC, IAPP, and Aβ were co-incubated with the enriched fraction for 14 days, dried onto grids, and stained with uranyl acetate. In the case of LC and Aβ, the affinity-purified IgG antibodies totally blocked fibril formation (not illustrated). For IAPP, electron micrographs revealed less ordered, dense aggregates characteristic of the assembly intermediate (O'Naullain et al., 2004), whereas, in the presence of unfractionated antibody or PBS, these preparations contained ultrastructurally typical fibrillar material (FIG. 16E). Further, purified IgG limited growth of LC, Aβ and IAPP fibrils in a dose-dependent fashion, with IC50 values in the extension assays of 1.6 FM, 0.05 μM, and 0.9 μM, respectively (FIGS. 16F-H).

The purified IgG antibodies also recognized amyloid in tissue, as documented immunohistochemically. These molecules immunostained the green birefringent congophilic deposits present in the renal glomeruli, pericardium, ovary, myocardium, and pancreas from patients with ALκ, ALλ, AA, ATTR, and AIAPP amyloidosis, respectively, as well as Aβ-containing cerebral cortical plaques in brain tissue obtained from an individual with Alzheimer's disease. This reactivity was totally abolished when the enriched antibodies were absorbed with LC fibrils (FIG. 17). Further, there was little or no staining with the residual fraction.

To determine if the fibril-reactive antibodies would have in vivo activity, radiolabeled affinity-purified IgG was used in imaging studies utilizing the murine amyloidoma model (Hrncic et al., 2000). Sets of 3 BALB/c mice were given 15 μg (˜160 μCi, ˜6 MBq) injections into the tail vein of ¹²⁵I-labeled enriched IgG one week after induction of amyloidomas containing 50 mg of human AL or ATTR extracts, were euthanized 72 hours later (non-amyloidoma bearing animals served as controls), and scanned by high-resolution microSPECT (Paulus et al., 2000). Localization of the radioiodinated antibodies to the amyloid was apparent in co-registered microSPECT/microCT images that revealed the radioisotope to be concentrated within the AL (FIG. 10) and ATTR amyloidomas (not illustrated), with only minimal background activity occurring in other organs or tissues. The preferential binding of the radioiodinated IgG antibodies with the amyloid also was indicated by the biodistribution of the labeled reagent, where the specific activities of the harvested tumors was ˜24% of the injected dose per g of tissue, a value at least 2 times greater than found at any other site and significantly more than that of skin, muscle, and fat (p<0.05) (Table 2). In contrast, the uptake of an irrelevant radiolabeled IgG monoclonal protein (MOPC 31-C) was <1%. The presence of radioiodinated antibody in other areas was comparable to that of a non-amyloidoma-bearing mouse.

TABLE 2
Biodistribution of radiolabeled enriched IGIV
in amyloidoma and non-amyloidoma-bearing mice

| | Enriched IGIV | | | | | |
|---|---|---|---|---|---|---|
| | AL^{a} | | ATTR^{a} | | Control^{b} | |
| | Average (%) | SD | Average (%) | SD | Average (%) | SD |
| skin | 3.1^{c} | 0.5 | 2.7^{c} | 0.6 | 3.2^{c} | 0.2 |
| muscle | 2.0 | 0.1 | 1.9 | 0.4 | 2.4 | 0.4 |
| fat | 2.1 | 0.6 | 1.5 | 0.5 | 2.1 | 0.5 |
| liver | 11.0 | 1.4 | 8.9 | 1.5 | 11.0 | 2.3 |
| spleen | 10.7 | 0.0 | 9.9 | 0.6 | 11.6 | 1.3 |
| pancreas | 4.0 | 0.1 | 3.6 | 0.1 | 4.8 | 1.3 |
| kidney | 8.9 | 0.0 | 7.3 | 1.0 | 10.1 | 1.4 |
| heart | 11.8 | 2.3 | 8.1 | 3.9 | 9.8 | 1.8 |
| amyloidoma | 23.5 | 2.0 | 24.8 | 14.3 | — | — |

^{a}Amyloidoma-bearing mice
^{b}Non-amyloidoma-bearing mice
^{c}Numbers represent an average of the specific activity of the injected dose of labeled antibody per g of tissue

Pairs of SCID mice bearing 100-mg, s.c. amyloidomas composed of ALκ, ALλ, or ATTR extracts were given, on days 0, 2, 4, 6, 8, and 10, a series of 0.25 ml s.c. injections of unfractionated or enriched IGIV (1 mg/ml) in each flank (another group received an equivalent amount of PBS alone). Eighteen days after the last injection, the ALκ, ALλ, and ATTR amyloidomas in animals that received the unfractionated IGIV were reduced in size 35, 64, and 30%, respectively, compared to those that were given PBS alone. This effect was more pronounced (63, 99, and 65%) with the enriched IGIV preparation (FIG. 1). The mean of tumor weights in the enriched, unfractionated, and PBS control mice was 0.14±0.09, 0.22±0.07, and 0.40±0.17, respectively, and a pairwise comparison of the extent of amyloidolysis revealed a significant difference between the enriched and control groups (p<0.05). As noted in studies with the amyloid-reactive 11-1F4 mAb (Hrncic et al., 2000), pronounced infiltration of activated neutrophils, mononuclear cells, and macrophages was seen in the resolving amyloidomas of the IgG-treated animals. Microscopic examination of Congo red-stained tissues showed that green birefringent material in both groups was confined to the amyloidomas and was not found in any other site.

### Discussion

The transformation of soluble proteins and peptides into insoluble amyloid fibrils reflects a series of conformational alterations that involve formation of amyloidogenic intermediates; self-association and stabilization of these components through interactions between β-sheets that lead to protofilaments/protofibrils; and, finally, interaction of the components to form the mature fibril (Serpell, 2000; Dobson, 2004; Makin et al., 2005). The profound structural changes that occur as normally folded globular proteins make this transition are manifested by differential epitope expression (Dumoulin et al., 2004; Glabe, 2004); e.g., one class of antibody may recognize sequence-specific linear epitopes exposed on the fibril, as well as the partially unfolded amyloidogenic intermediate and native precursor protein; a different type reacts with neoepitopes present on the fibril and assembly intermediates (i.e., antigenic regions buried in the native molecule which become exposed as a result of protein unfolding); and yet another binds to a conformational epitope present on all fibrils, irrespective of primary structure.

Monoclonal antibodies representative of groups 1, 2, and 3 have been generated against Aβ, TTR, and LC fibrils and are exemplified by reagents that recognize, respectively, a site within the exposed N-terminal six residues of the native and fibrillar forms of Aβ (Solomon et al., 1996), a neoepitope on TTR (Goldsteins et al., 1999), and a common fibril-specific epitope (Hrncic et al., 2000; O'Nuallain et al., 2002). Notably, the polyclonal fibril-reactive IgG antibodies used in the studies contain molecules representative of group 3 that specifically recognize a generic epitope expressed by fibrils formed from at least five different amyloidogenic precursors—LC, SAA, TTR, IAPP, and Aβ—but do not react with these proteins or peptides in their native states. In this respect, they differ from, for example, the anti-Aβ reactive components found in human sera (Hyman et al., 2001; Weksler et al., 2002; Du et al., 2001), an immune globulin product (Dodel et al., 2002; Du et al., 2003; Dodel et al., 2004), the IgM mAbs generated from peripheral blood lymphocytes (Geylis et al., 2005), and other types of anti-Aβ reagents (Liu et al., 2004). While other investigators have identified antibodies that differentiate between the soluble and fibrillar configurations of Aβ (Hock et al. 2002; Bard et al. 2003), it remains to be established if these reagents bind heterologous fibrils as does IGIV.

The amyloid-reactive IgGs that we have identified in human sera are unlike other polyreactive antibodies (Notkins, 2004) in that they are fibril-specific; further they do not bind non-amyloid aggregated or fibrillar macromolecules. These components seemingly represent yet another example of auto-reactive molecules formed as part of a humoral immune response to an endogenous or exogenous antigenic stimulus. One intrinsic source of fibrils may be those formed from normally soluble proteins that have the propensity to self-associate and become fibrillogenic as part of the aging process (Enqvist et al., 2003). Alternatively, extrinsic fibrillar materials (Niewold et al., 1987; Xing et al., 2001; Lundmark et al., 2002; Kisilevsky et al., 1999; Chapman et al., 2002; Glover et al., 1997; Mackay et al., 2001; Lundmark et al., 2005) may themselves be immunogenic or, as fibrillogenesis is a nucleation-dependent process (Harper et al., 1997), serve as seeds to generate fibrils from amyloidogenic precursor proteins.

The functional significance of IgG-associated pan fibril-reactive antibodies is presently unknown. As shown in fibril formation and extension assays, these molecules can act as inhibitors of fibrillogenesis. Further, such antibodies may facilitate destruction of amyloid deposits or clear fibrillar aggregates from the circulation, analogous to the removal of misfolded intracellular proteins by molecular chaperones.

As yet, there are only limited methods (Hawkins et al., 1990; Nordberg, 2004) to document radiographically the extent or presence of amyloid deposition (or its resolution); thus, there is a critical need for an objective means to ascertain a patient's response to treatment and/or to determine if relapse has occurred. Radiolabeled fibril-reactive antibodies are useful as imaging agents, as evidenced by the selective uptake of radioiodinated fibril-affinity purified IgG by human AL and ATTR amyloidomas. Further, the distribution data, co-registered SPECT/CT images, and the favorable signal-to-noise ratio have indicated the diagnostic potential of this reagent for patients with systemic forms of amyloidosis. For those with Alzheimer's disease, fibril-reactive, single chain Fv components may be useful because their lower mol wt would facilitate passage into the brain.

Currently, there are few therapeutic options available for patients with amyloid-associated disease. Thus, the use of fibril-reactive IgG antibodies of human origin to facilitate removal of amyloid deposits may prove to be an effective means of treatment. In this regard, the results show that the low titers of fibril-reactive antibodies found in the sera of patients with AL or ATTR amyloidosis could be increased significantly when enriched IgG was added to the specimens (O'Nuallain, et al., unpublished data). This enhanced reactivity was evidenced, not only against LC or TTR fibrils, but also to protein extracted from the patient's own amyloid-laden tissue. Based on the experimental results, amyloid resolution resulted from an in situ 3-step process that included: (1) the binding or opsonization of fibrils by the fibril-reactive IgG antibodies; (2) attraction and activation of neutrophils and macrophages via Fc receptor interaction; and (3) enzymatic and/or chemical proteolysis of the amyloid by endopeptidases or reactive oxygen species, respectively.

The potential of passive immunotherapy to effect amyloidolysis also has been evidenced in transgenic murine models of Alzheimer's disease (Bard et al., 2003; Dodel et al., 2003; Schenk et al., 2004). Since the antibodies administered reacted with the AD monomer (as in the case of those generated via vaccination with Aβ-related peptides [Schenk et al., 2004]), other investigators have attributed this response to the binding and/or sequestration of native Aβ (DeMattos et al., 2001). Given that fibril-reactive IgG does not react with this or other non-fibrillar forms of amyloidogenic precursor molecules, infusion of these antibodies could be clinically advantageous since it is unlikely that potentially harmful immune complexes would be formed.

In summary, high-affinity antibodies have been isolated from human pooled immune globulin that specifically recognize fibrils formed from five different amyloidogenic precursors. Their binding specificity have been documented through the use of a sensitive fluoroimmunoassay, as well as immunohistochemically where they immunostained amyloid deposits in tissue. Further, these molecules could inhibit fibrillogenesis, as shown in fibril formation and extension assays. In other studies involving an in vivo experimental murine model, the purified antibodies, when radiolabeled, served as diagnostic reagents capable of imaging amyloid deposits. Moreover, therapeutic activity was evidenced by their acceleration of amyloidolysis in animals bearing human amyloidomas. Based on our experimental results, fibril-reactive IgG antibodies may provide a novel diagnostic and therapeutic modality to improve the invariably poor prognoses of patients with amyloid-associated disease.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference in their entirety.

### REFERENCES

The following references are herein incorporated by reference in their entity for all purposes:
Bard F, Barbour R, Cannon C, et al. Epitope and isotype specificities of antibodies to β-amyloid peptide for protection against Alzheimer's disease-like neuropathology. Proc Natl Acad Sci USA 100:2023-2028, 2003.
Bard F, Cannon C, Barbour R, et al. Peripherally administered antibodies against amyloid β-peptide enter the central nervous system and reduce pathology in a mouse model of Alzheimer disease. Nat Med 6:916-919, 2000.
Bard, F., R. Barbour, C. Cannon, R. Carretto, M. Fox, D. Games, T. Guido, K. Hoenow, K. Hu, K. Johnson-Wood, K. Khan, D. Kholodenko, C. Lee, M. Lee, R. Motter, M. Nguyen, A. Reed, D. Schenk, P. Tang, N. Vasquez, P. Seubert, and T. Yednock. Epitope and isotype specificities of antibodies to beta-amyloid peptide for protection against Alzheimer's disease-like neuropathology. PNAS. 100: 2023-2028, 2003.
Benson M D. Amyloidosis. The Metabolic and Molecular Bases of Inherited Disease: In: Beaud et al, Sly W S and Valle D (eds.) 8^{th} ed., pp. 5345-5378, 2001.
Brendza R P, Bacskai B J, Cirrito J R, et al. Anti-Aβ antibody treatment promotes the rapid recovery of amyloid-associated neuritic dystrophy in PDAPP transgenic mice. J Clin Invest 115:428-433, 2005.
Bussiere T, Bard F, Barbour R, et al. Morphological characterizations of thioflavin-S-positive amyloid plaques in transgenic Alzheimer mice and effect of passive Aβimmunotherapy on their clearance. Am J Pathol 165:987-995, 2004.
Chapman, M. R., A. S. Kowal, E. C. Schirmer, M. M. Patino, J. J. Liu, and S. Lindquist. Role of Escherichia coli curli operons in directing amyloid fiber formation. Science. 295: 851-855, 2002.
Curin-Serbec V, Bresjanac M, Popovic M, et al. Monoclonal antibody against a peptide of human prion protein discriminates between Creutzfeldt-Jacob's disease-affected and normal brain tissue. J Biol Chem 279:3694-3698, 2004.
DeMattos R B, Bales K R, Cummins D J, Dodart J C, Paul S M, and Holtzman D M. Peripheral anti-Aβ antibody alters CNS and plasma Aβ clearance and decreases brain Aβburden in a mouse model of Alzheimer's disease. Proc Natl Acad Sci USA 98:8850-8855, 2001.
Diamandis E P. Immunoassays with time-resolved fluorescence spectroscopy: principles and applications. Clin Biochem 21:139-150, 1988.
Dobson, C. M. Experimental investigation of protein folding and misfolding. Methods. 34: 4-14, 2004.
Dodart J C, Bales K R, Gannon K S, et al. Immunization reverses memory deficits without reducing brain Aβ burden in Alzheimer's disease model. Nat Neurosci 5:452-457, 2002.
Dodel R, Hampel H, Depboylu C, et al. Human antibodies against amyloid p peptide: a potential treatment for Alzheimer's disease. Ann Neurol 52:253-256, 2002.
Dodel, R C, H. Hampel, and Y. Du. Immunotherapy for Alzheimer's disease. Lancet. 2: 215-220, 2003.
Dodel, R. C., Y. Du, C. Depboylu, H. Hampel, L. Frolich, A. Haag, U. Hemmeter, S. Paulsen, S. J. Teipel, S. Brettschneider, A. Spottke, C. Nolker, H. J. Moller, X. Wei, M. Farlow, N. Sommer, and W. H. Oertel. Intravenous immunoglobulins containing antibodies against α-amyloid for the treatment of Alzheimer's disease. J. Neurol. Neurosurg. Psychiatry. 75: 1472-1474, 2004.
Dodel, R., H. Hampel, C. Depboylu, S. Lin, F. Gao, S. Schock, S. Jackel, X. Wei, K. Buerger, C. Höft, B. Hemmer, H. J. Moller, M. Farlow, W. H. Oertel, N. Sommer, and Y. Du. Human antibodies against amyloid β peptide: a potential treatment for Alzheimer's disease. Ann. Neurol. 52: 253-256, 2002.
Du Y, Dodel R, Hampel H, et al. Reduced levels of amyloid β-peptide antibody in Alzheimer disease. Neurology 57:801-805, 2001.
Du Y, Wei X, Dodel R, et al. Human anti β-amyloid antibodies block β-amyloid fibril formation and prevent β-amyloid induced neurotoxicity. Brain 126:1935-1939, 2003.
Dumoulin M and Dobson C M. Probing the origins, diagnosis and treatment of amyloid diseases using antibodies. Biochimie 86:589-600, 2004.
Enqvist S, Peng S, Persson A, and Westermark P. Senile amyloidoses-diseases of increasing importance. Acta Histochem 105:377-378, 2003.
Fang Q, Kannapell C C, Fu S M, Xu S, and Gaskin F. V_{H} and V_{L} gene usage by anti-β-amyloid autoantibodies in Alzheimer's disease: detection of highly mutated V regions in both heavy and light chains. Clin Immunol Immunopath 75:159-167, 1995.
Franklin E C and Zucker-Franklin D. Antisera specific for human amyloid reactive with conformational antigens. Proc Soc Exp Biol Med 140:565-568, 1972.
Friguet B, Djavadi-Ohaniance L, and Goldberg Me. Some monoclonal antibodies raised with a native protein bind preferentially to the denatured antigen. Mol Immunol 21:673-677, 1984.
Gaskin F, Finley J, Fang Q, Xu S, and Fu S M. Human antibodies reactive with β-amyloid protein in Alzheimer's disease. J Exp Med 177:1181-1186, 1993.
Gevorkian G, Petrushina I, Manoutcharian K, et al. Mimotopes of conformational epitopes in fibrillar β-amyloid. J Neuroimmunol 156:10-20, 2004.
Geylis V, Kourilov V, Meiner Z, Nennesmo I, Bogdanovic N, and Steinitz M. Human monoclonal antibodies against amyloid-beta from healthy adults. Neurobiol Aging 26:597-606, 2005.
Glabe C G., Conformation-dependent antibodies target diseases of protein misfolding. Trends Biochem Sci 29:542-547, 2004. Glover, J. R., A. S. Kowal, E. C. Schirmer, M. M. Patino, J. J. Liu, and S. Lindquist. Self-seeded fibers formed by Sup35, the protein determinant of [PSI+], a heritable prion-like factor of S. cerevisiae. Cell. 89: 811-819, 1997.
Goldsbury C S, Cooper G J, Goldie K N, et al. Polymorphic fibrillar assembly of human amylin. J Struct Biol 119:17-27, 1997.
Goldsteins G, Persson H, Andersson K, et al. Exposure of cryptic epitopes on transthyretin only in amyloid and in amyloidogenic mutants. Proc Natl Acad Sci USA 96:3108-3113, 1999.
Harper, J. D., and P. T. Lansbury, Jr. Models of amyloid seeding in Alzheimer's disease and scrapie: mechanistic truths and physiological consequences of the time-dependent solubility of amyloid proteins. Annu. Rev. Biochem. 66: 385-407, 1997.
Hawkins, P. N., J. P. Lavender, and M. B. Pepys. Evaluation of systemic amyloidosis by scintigraphy with ¹²³I-labeled serum amyloid P component. N. Engl. J. Med. 323: 508-513, 1990.
Higham C E, Jaikaran E T, Fraser P E, Gross M, and Clark A. Preparation of synthetic human islet amyloid polypeptide (IAPP) in a stable conformation to enable study of conversion to amyloid-like fibrils. FEBS Letters 470:55-60, 2000.
Hock, C., U. Konietzko, A. Papassotiropoulos, A. Wollmer, J. Streffer, R. C. von Rotz, G. Davey, E. Moritz, and R. M. Nitsch. Generation of antibodies specific for beta-amyloid by vaccination of patients with Alzheimer disease. Nat. Med. 8: 1270-1275, 2002.
Höök F, Rodahl M, Kasemo B, and Brzezinski P. Structural changes in hemoglobin during adsorption to solid surfaces: effects of pH, ionic strength, and ligand binding. Proc Natl Acad Sci USA 95:12271-12276, 1998.
Horikoshi Y, Mori T, Maeda M, Kinoshita N, Sato K, and Yamaguchi H. Aβ N-terminal-end specific antibody reduced β-amyloid in Alzheimer-model mice. Biochem Biophys Res Commun 325:384-387, 2004.
Hrncic R, Wall J, Wolfenbarger D A, et al. Antibody-mediated resolution of light chain-associated amyloid deposits. Am J Pathol 157:1239-1246, 2000.
Hyman B T, Smith C, Buldyrev I, et al. Autoantibodies to amyloid-β and Alzheimer's disease. Ann Neurol 49:808-810, 2001.
Kayed R, Head E, Thompson J L, et al. Common structure of soluble amyloid oligomers implies common mechanism of pathogenesis. Science 300:486-489, 2003.
Kennel S J, Foote L J, Lankford P K, Johnson M, Mitchell T, and Braslawsky G R. Direct binding of radioiodinated monoclonal antibody to tumor cells: significance of antibody purity and affinity for drug targeting or tumor imaging. Hybridoma 2:297-310, 1983.
Kisilevsky, R., L. Lemieux, L. Boudreau, D.-S. Yang, and P. Fraser. New clothes for amyloid enhancing factor (AEF): silk as AEF. Amyloid. 6: 98-106, 1999.
Lai Z, Colón W, and Kelly J W. The acid-mediated denaturation pathway of transthyretin yields a conformational intermediate that can self-assemble into amyloid. Biochemistry 35:6470-6482, 1996.
Landsbury P T Jr, Costa P R, Griffiths J M, et al. Structural model for the beta-amyloid fibril based on interstrand alignment of an antiparallel-sheet comprising a C-terminal peptide. Nat Struct Biol 2:990-998, 1995.
LeVine H I I I. Thioflavine T interaction with amyloid β-sheet structures. Amyloid: Int J Exp Clin Invest 2:1-6, 1995.
Linke R P, Zucker-Franklin D, and Franklin E C. Morphologic, chemical and immunologic studies of amyloid-like fibrils formed from Bence Jones proteins by proteolysis. J Immunol 111:10-23, 1973.
Liu, R., B. Yuan, S. Emadi, A. Zameer, P. Schulz, C. McAllister, Y. Lyubchenko, G. Goud, and M. R. Sierks. Single chain variable fragments against β-amyloid (Aβ) can inhibit A, aggregation and prevent Aβ-induced neurotoxicity. Biochemistry. 43: 6959-6967, 2004.
Lundmark, K., G. T. Westermark, A. Olsen, and P. Westermark. Protein fibrils in nature can enhance amyloid protein A amyloidosis in mice: cross-seeding as a disease mechanism. Proc. Natl. Acad. Sci. USA. 102: 6098-6102, 2005.
Lundmark, K., G. T. Westermark, S, Nyström, C. L. Murphy, A. Solomon, and P. Westermark. Transmissibility of systemic amyloidosis by a prion-like mechanism. Proc. Natl. Acad. Sci. USA. 99: 6979-6984, 2002.
Mackay, J. P., J. M. Matthews, R. D. Winefield, L. G. Mackay, R. G. Haverkamp, and M. D. Templeton. The hydrophobin EAS is largely unstructured in solution and functions by forming amyloid-like structures. Structure. 9: 83-91, 2001.
Makin O S, Atkins E, Sikorski P, Johansson J, and Serpell L C. Molecular basis for amyloid fibril formation and stability. Proc Natl Acad Sci USA 102:315-320, 2005.
Meehan S, Berry Y, Luisi B, Dobson C M, Carver J A, and MacPhee C E. Amyloid fibril formation by lens crystallin proteins and its implications for cataract formation. J Biol Chem 279:3413-3419, 2004.
Morley J E, Farr S A, and Flood J F. Antibody to amyloid β protein alleviates impaired acquisition, retention, and memory processing in SAMP8 mice. Neurobiol Learn Mem 78:125-138, 2002.
Murphy C L, Eulitz M, Hrncic R, et al. Chemical typing of amyloid protein contained in formalin-fixed paraffin-embedded biopsy specimens. Am J Clin Pathol 116:135-142, 2001.
Naiki H and Gejyo F. Kinetic analysis of amyloid fibril formation. Methods Enzymol 309:305-318, 1999.
Niewold, T. A., P. R. Hol, A. C. van Andel, E. T. Lutz, and E. Gruys. Enhancement of amyloid induction by amyloid fibril fragments in hamster. Lab. Invest. 56: 544-549, 1987.
Nordberg, A. PET imaging of amyloid in Alzheimer's disease. Lancet Neurol. 3: 519-527, 2004.
Notkins, A. L. Polyreactivity of antibody molecules. Trends Immunol. 25: 174-179, 2004.
O'Nuallain B and Wetzel R. Conformational Abs recognizing a generic amyloid fibril epitope. Proc Natl Acad Sci USA 99:1485-1490, 2002.
O'Nuallain B, Murphy C L, Wolfenbarger D A, Kennel S, Solomon A, and Wall J S. The amyloid-reactive monoclonal antibody 11-1F4 binds a cryptic epitope on fibrils and partially denatured immunoglobulin light chains and inhibits fibrillogenesis. In: Grateau G, Kyle R A, Skinner M (eds.) Amyloid and Amyloidosis. Proceedings of the Xth International Congress on Amyloidosis. CRC Press. pp. 482-484, 2004.
O'Nuallain B, Williams A D, Westermark P, and Wetzel R. Seeding specificity in amyloid growth induced by heterologous fibrils. J Biol Chem 279:17490-17499, 2004.
Paramithiotis E, Pinard M, Lawton T, et al. A prion protein epitope selective for the pathologically misfolded conformation. Nat Med 9:893-899, 2003.
Paulus M J, Gleason S S, Kennel S J, Hunsicker P R, and Johnson D K. High resolution X-ray computed tomography: an emerging tool for small animal cancer research. Neoplasia 2:62-70, 2000.
Ross C A and Poirier M A. Protein aggregation and neurodegenerative disease. Nat Med 10:S10-S17, 2004.
Schenk, D., M. Hagen, and P. Seubert. Current progress in beta-amyloid immunotherapy. Curr. Opin. Immunol. 16: 599-606, 2004.
Serpell L C. Alzheimer's fibrils: structure and assembly. Bichim Biophys Acta 1502:16-30, 2000.

Seshadri S, Khurana R, and Fink A L. Fourier transform infrared spectroscopy in analysis of protein deposits. Methods Enzymol 309:559-576, 1999.
Solomon A, Weiss D T, and Wall J S. Immunotherapy in systemic primary (AL) amyloidosis using amyloid-reactive monoclonal antibodies. Cancer Biother Radiopharm 18:853-860, 2003.
Solomon A. Light chains of immunoglobulins. Methods Enzymol 116:101-121, 1985. Solomon B, Koppel R, Frankel D, and Hanan-Aharon E. Disaggregation of Alzheimer β-amyloid by site-directed mAb. Proc Natl Acad Sci USA 94:4109-4112, 1997.
Solomon B, Koppel R, Hanan E, and Katzav T. Monoclonal antibodies inhibit in vitro fibrillar aggregation of the Alzheimer β-amyloid peptide. Proc Natl Acad Sci USA 93:452-455, 1996.
Wall J, Schell M, Hrncic R, et al. Treatment of amyloidosis using an anti-fibril monoclonal antibody: Pre-clinical efficacy in a murine model of AA-amyloidosis. In: Bely M, Apathy A, (eds.) Amyloid and Amyloidosis. The Proceedings of the IX International Symposium on Amyloidosis. Hungary, pp 158-160, 2001.
Wall J, Schell M, Murphy C, Hrncic R, Stevens F J, and Solomon A. Thermodynamic instability of human λ6 light chains: correlation with fibrillogenicity. Biochemistry 38:14101-14108, 1999.
Wall J S, Paulus M J, Kennel S J, et al. Radioimaging of primary (AL) amyloidosis with an amyloid-reactive monoclonal antibody. In: Grateau G, Kyle R A, Skinner M, (eds.) Amyloid and Amyloidosis. Proceedings of the Xth International Congress on Amyloidosis. CRC Press pp. 37-39, 2004.
Walrand S H, van Elmbt L R, and Pauwels S. A non-negative fast multiplicative algorithm in 3D scatter-compensated SPET reconstruction. Eur J Nucl Med 23:1521-1526, 1996.
Weksler M E, Relkin N, Turkenich R, LaRusse S, Zhou L, and Szabo P. Patients with Alzheimer disease have lower levels of serum anti-amyloid peptide antibodies then healthy elderly individuals. Exp Gerontol 37:943-948, 2002.
Westermark G T, Johnson K H, and Westermark P. Staining methods for identification of amyloid in tissue. Methods Enzymol 309:3-25, 1999.
Westermark P, Benson M D, Buxbaum J N, et al. Amyloid fibril nomenclature—2002. Amyloid J Protein Folding Disord 9:197-200, 2002.
Westermark, P., M. D. Benson, J. N. Buxbaum, A. S. Cohen, B. Frangione, S. Ikeda, C. L. Masters, G. Merlini, M. J. Saraiva, and J. D. Sipe. Amyloid: Toward terminology clarification. Amyloid. 12:1-4, 2005.
Wilcock D M, Rojiani A, Rosenthal A, et al. Passive amyloid immunotherapy clears amyloid and transiently activities microglia in a transgenic mouse model of amyloid deposition. J Neurosci 24:6144-6151, 2004.
Xing, Y., A. Nakamura, T. Chiba, K. Kogishi, T. Matsushita, F. Li, Z. Guo, M. Hosokawa, M. Mori, and K. Higuchi. Transmission of mouse senile amyloidosis. Lab. Invest. 81: 493-499, 2001.
Zagorski M G, Yang J, Shao H, Ma K, Zeng H, and Hong A. Methodological and chemical factors affecting amyloid-β peptide amyloidogenicity. Methods Enzymol 309:189-204, 1999.

1-20. (canceled)

21: A pharmaceutical composition comprising an effective amount of enriched IGIV or a fragment thereof formulated for binding amyloid deposits or for binding partially denatured amyloidogenic precursor proteins.

22. (canceled)

23: The composition of claim 21, wherein the enriched IGIV or a fragment thereof comprises about a 10%, 20%, 50%, 75%, 100%, 200%, 400% or more increase in binding compared to the starting material.

24: The composition of claim 21, wherein the enriched IGIV or a fragment thereof comprises about a 2-fold, 3-fold, 4-fold, 5-fold, 7-fold, 10-fold, 20-fold, 50-fold, 100-fold, 500-fold or more binding compared to the starting material.

25: The composition of claim 21, wherein the enriched IGIV or a fragment thereof comprises about 1%, 5%, 10%, 25%, 50%, 75%, 80% or more fibril reactive antibodies.

26. (canceled)

27: The composition of claim 21, wherein the IGIV is mammalian IGIV.

28: The of composition of claim 27, wherein the mammal is selected from the group consisting of human, mouse, rat, hamster, guinea pig, dog, cat, rabbit, pig, goat, sheep, cow, chimpanzee and monkey.

29: A method of treating a subject having amyloid deposition comprising administering to the subject a therapeutically effective amount of the pharmaceutical composition of claim 21, wherein the IGIV or fragment thereof binds an amyloid fibril or partially denatured amyloidogenic precursor polypeptide.

30: A method of removing amyloid deposits from a subject comprising administering to the subject a therapeutically effective amount of the pharmaceutical composition of claim 21 to remove amyloid deposits, and allowing the IGIV or the fragment thereof to bind amyloid deposits, thereby removing the amyloid deposit from the subject.

31: A method of inhibiting formation of amyloid deposits in a subject comprising administering to the subject a therapeutically effective amount of the pharmaceutical composition of claim 21 to inhibit formation of amyloid deposits, and allowing the IGIV or fragment thereof to bind amyloid-forming precursor protein, thereby inhibiting the formation of amyloid deposits.

32: A method of modulating formation of amyloid deposits in a subject comprising administering to the subject a therapeutically effective amount of the pharmaceutical composition of claim 21 to modulate formation of amyloid deposits, and allowing the IGIV or fragment thereof to bind an amyloid fibril or partially denatured amyloidogenic precursor polypeptide, thereby modulating formation of amyloid deposits.

33: A method of detecting amyloid deposits in a subject comprising administering to the subject a therapeutically effective amount of the pharmaceutical composition of claim 21 to detect amyloid deposits and allowing the IGIV or fragment thereof to bind amyloid deposits, and detecting amyloid deposits.

34: A method of imaging amyloid deposits in a subject comprising administering to the subject a therapeutically effective amount of the pharmaceutical composition of claim 21 to image amyloid deposits and allowing the IGIV or fragment thereof to bind amyloid deposits, and obtaining an image of the amyloid deposits.

35: The method of claim 33 or 34, wherein the IGIV is tagged with an agent selected from the group consisting of radionuclides, contrasting agents, enzymes, dyes.

36: The method of claim 34, wherein the amyloid deposits are imaged by SPECT, CT, PET, x-ray, MRI, optical or infrared imaging and ultrasound.

37: The method of any one of claims 29 to 34, wherein the amyloid deposits are composed of immunoglobulin light chains, transthyretin, Aβ peptide, islet amyloid precursor polypeptide or other amyloid-forming precursor protein.

38: A method of enriching a sample of IGIV comprising preparing an amyloid fibril-conjugated affinity column, loading an IGIV sample onto the column, and eluting enriched IGIV from the column.

39: The method of claim 38 comprising measuring the EC50 value of the IGIV samples purified from the column and isolating IGIV samples with EC50 values of 0.1 to 100 nM.

40: An affinity purification matrix comprising amyloid fibrils.
